(19) United States
(12) Patent Application Publication
(10) Pub. No.: US 2026/0345798 A1
(43) Pub. Date: Nov. 5, 2026

(54) COMPOUNDS AND METHODS OF USE THEREOF

(71) Applicant: Virginia Commonwealth University, Richmond, VA (US)

(72) Inventors: Paul B. Fisher, Richmond, VA (US); Swadesh K. Das, Moseley, VA (US)

(21) Appl. No.: 19/486,885

(22) PCT Filed: May 22, 2024
(86) PCT No.: PCT/US2024/030521

§ 371 Date: Nov. 21, 2025

Related U.S. Application Data
(60) Provisional application No. 63/503,696, filed on May 22, 2023.

(51) Int. Cl. A61K 47/62; A61K 39/00; A61K 39/395; A61K 47/54; A61P 35/04

(52) U.S. Cl. CPC A61K 47/62; A61K 39/3955; A61K 47/545; A61P 35/04; A61K 2039/505

(57) ABSTRACT

Provided herein are compounds and pharmaceutical compositions for treating cancer (e.g., melanoma), and methods of use thereof. The compounds disclosed herein inhibit MDA-9/Syntenin by binding both MDA-9/Syntenin PDZ domains.

### CROSS REFERENCE TO RELATED APPLICATIONS

This application claims the benefit of and priority to U.S. Provisional Patent Application No. 63/503,696, filed on May 22, 2023, the disclosure of which is hereby incorporated by reference in its entirety for all purposes.

### GOVERNMENT SUPPORT

This invention was made with government support under grant numbers CA244993 and P30 CA016059 awarded by the National Institutes of Health/National Cancer Institute. The government has certain rights in the invention.

### BACKGROUND

Worldwide, about 1.7% of all newly diagnosed cancers are melanoma resulting in approximately 55,500 deaths annually (1). As with other cancers, neoplastic development of melanoma correlates with acquisition of genetic/epigenetic alterations and activation of numerous oncogenic signals, e.g., mutations in BRAFV600E (Val600Glu) and the telomerase reverse-transcriptase promoter, loss of Cyclin Dependent kinase inhibitor 2A, mutations in phosphatase-and-tensin homolog (PTEN) or tumor protein 53 (TP53) (2,3). Despite significant advances in defining the genetic landscape of melanoma, this disease remains deadly after becoming metastatic, and there is a pressing mandate to identify both therapeutic targets and intervention strategies.

### SUMMARY

Provided herein are compounds and pharmaceutical compositions that are useful for treating cancer (e.g., melanoma), and methods of treating cancer (e.g., melanoma) using the compounds and pharmaceutical compositions described herein.

In one aspect, provided herein is a compound of formula (I),

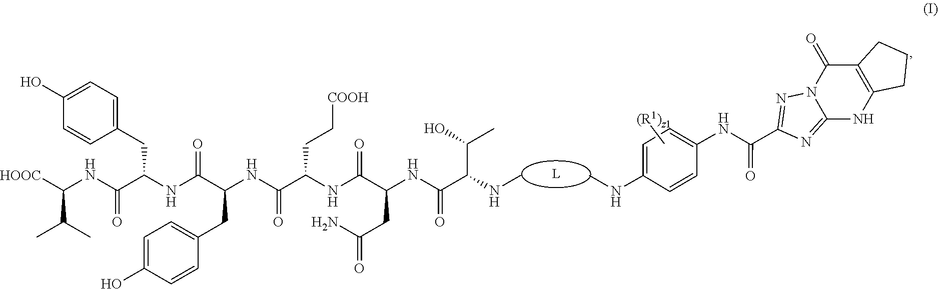

or a pharmaceutically acceptable salt thereof, wherein
L is a non-cleavable linker;
each R¹ is independently selected from halo, —OH, and C₁₋₆ alkyl; and
z1 is an integer from 0 to 3.

In some embodiments, each R¹ is independently halo or C₁₋₆ alkyl.

In some embodiments, z1 is 2.

In some embodiments, the compound is a compound of formula (Ia),

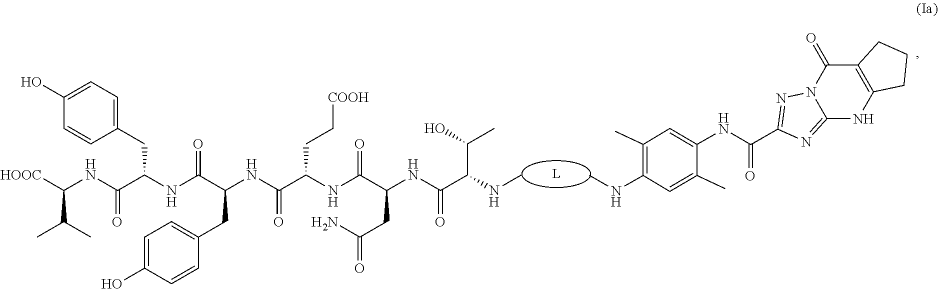 [SMILES: CN[C@H](C(=O)N[C@@H](CC(N)=O)C(=O)N[C@@H](CCC(=O)O)C(=O)N[C@@H](Cc1ccc(O)cc1)C(=O)N[C@@H](Cc1ccc(O)cc1)C(=O)N[C@H](C(=O)O)C(C)C)[C@@H](C)O ; CNc1cc(C)c(NC(=O)c2nc3[nH]c4c(c(=O)n3n2)CCC4)cc1C]

or a pharmaceutically acceptable salt thereof.

In some embodiments, L is at least 15 Angstroms in length.

In some embodiments, L is

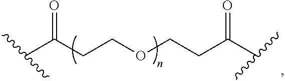

wherein n is an integer from 5 to 10.

In some embodiments, n is an integer from 5 to 7.

In another aspect, provided herein is a compound represented by the formula

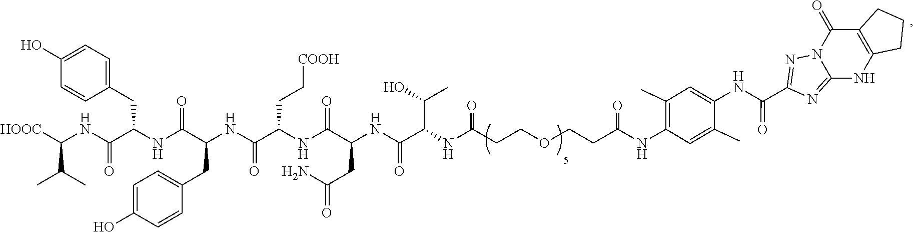

or a pharmaceutically acceptable salt thereof.

In another aspect, provided herein is a compound represented by the formula

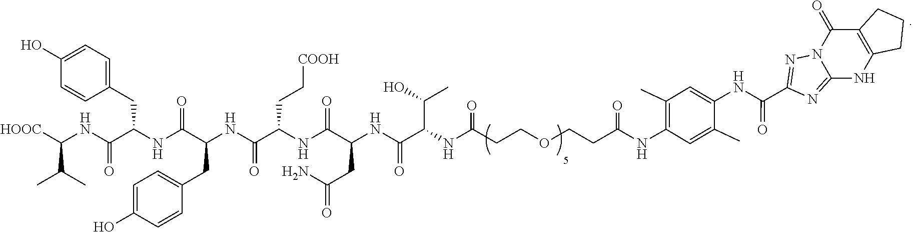

In another aspect, provided herein is a pharmaceutical composition comprising a compound disclosed herein (e.g., a compound of formula (I) or formula (Ia)) and one or more pharmaceutically acceptable excipients.

In another aspect, provided herein is a method of treating cancer in a subject in need thereof, the method comprising administering to the subject an effective amount of a compound or pharmaceutical composition disclosed herein.

In another aspect, provided herein is a method of treating cancer in a subject in need thereof, the method comprising administering to the subject an effective amount of a compound or pharmaceutical composition disclosed herein, and an effective amount of an α-PD-L1 checkpoint inhibitor. In some embodiments, the α-PD-L1 checkpoint inhibitor is selected from the group consisting of atezolizumab, avelumab, durvalumab, KN035, cosibelimab, AUNP12, CA-170, and BMS-986189. In some embodiments, the α-PD-L1 checkpoint inhibitor is selected from the group consisting of atezolizumab, avelumab, and durvalumab.

In some embodiments, the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma. In some embodiments, the cancer is melanoma. In some embodiments, the melanoma is metastatic melanoma. In some embodiments, treating the cancer comprises preventing or slowing the development of metastasis of the cancer. In some embodiments, treating melanoma comprises preventing or slowing the development of melanoma lung metastases.

In some embodiments, the melanoma comprises a p53 mutation. In some embodiments, the melanoma does not comprise a p53 mutation.

In some embodiments, the treating comprises reducing nuclear factor kappa-light-chain-enhancer of activated B cells (NF-κB) signaling in the cancer by administering to the subject a compound or pharmaceutical composition disclosed herein. In some embodiments, the treating comprises reducing matrix metalloproteinase 2 (MMP-2) and/or matrix metalloproteinase 9 (MMP-9) expression in the cancer by administering to the subject a compound or pharmaceutical composition disclosed herein. In some embodiments, the treating comprises reducing insulin growth factor binding protein-2 (IGFBP-2) expression in the cancer by administering to the subject a compound or pharmaceutical composition disclosed herein. In some embodiments, the treating comprises reducing interleukin-6, interleukin-10, and/or interleukin-16 expression in the cancer by administering to the subject a compound or pharmaceutical composition disclosed herein. In some embodiments, the treating comprises increasing IFN-γ-producing CD8⁺ T cell populations in the subject by administering to the subject a compound or pharmaceutical composition disclosed herein. In some embodiments, the treating comprises decreasing myeloid-derived suppressor cell (MDSC) populations in the subject by administering to the subject a compound or pharmaceutical composition disclosed herein.

In another aspect, provided herein is a method of preventing or slowing the development of cancer metastasis in a subject with cancer, the method comprising administering to the subject an effective amount of a compound or pharmaceutical composition disclosed herein.

In another aspect, provided herein is a method of preventing or slowing the development of cancer metastasis in a subject with cancer, the method comprising administering to the subject an effective amount of a compound or pharmaceutical composition disclosed herein, and an effective amount of an α-PD-L1 checkpoint inhibitor. In some embodiments, the α-PD-L1 checkpoint inhibitor is selected from the group consisting of atezolizumab, avelumab, durvalumab, KN035, cosibelimab, AUNP12, CA-170, and BMS-986189. In some embodiments, the α-PD-L1 checkpoint inhibitor is selected from the group consisting of atezolizumab, avelumab, and durvalumab.

In some embodiments, the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma. In some embodiments, the cancer is melanoma. In some embodiments, the melanoma is metastatic melanoma. In some embodiments, preventing or slowing the development of melanoma metastasis comprises preventing or slowing the development of melanoma lung metastases.

In some embodiments, the melanoma comprises a p53 mutation. In some embodiments, the melanoma does not comprise a p53 mutation.

In some embodiments, the preventing or slowing the development of the cancer comprises reducing nuclear factor kappa-light-chain-enhancer of activated B cells (NF-κB) signaling in the cancer by administering to the subject a compound or pharmaceutical composition disclosed herein. In some embodiments, the preventing or slowing the development of the cancer comprises reducing matrix metalloproteinase 2 (MMP-2) and/or matrix metalloproteinase 9 (MMP-9) expression in the cancer by administering to the subject a compound or pharmaceutical composition disclosed herein. In some embodiments, the preventing or slowing the development of the cancer comprises reducing insulin growth factor binding protein-2 (IGFBP-2) expression in the cancer by administering to the subject a compound or pharmaceutical composition disclosed herein. In some embodiments, the preventing or slowing the development of the cancer comprises reducing interleukin-6, interleukin-10, and/or interleukin-16 expression in the cancer by administering to the subject a compound or pharmaceutical composition disclosed herein. In some embodiments, the preventing or slowing the development of the cancer comprises increasing IFN-γ-producing CD8⁺ T cell populations in the subject by administering to the subject a compound or pharmaceutical composition disclosed herein. In some embodiments, the preventing or slowing the development of the cancer comprises decreasing myeloid-derived suppressor cell (MDSC) populations in the subject by administering to the subject a compound or pharmaceutical composition disclosed herein.

### BRIEF DESCRIPTION OF THE DRAWINGS

FIG. 1A is the structural formula of IVMT-Rx-3.

FIG. 1B is an image showing the docked structure of IVMT-Rx-3 in complex with MDA-9/Syntenin.

FIG. 1C is a binding curve of IVMT-Rx-3 bound to MDA-9/Syntenin. The binding constant Ka was measured from the binding curve generated through a microscale thermophoresis (MST) assay.

FIG. 1D is a graph showing the mean plasma concentration of IVMT-Rx-3 in mice administered IVMT-Rx-3 intravenously and intraperitoneally.

FIG. 2A is a histogram showing the results of an MTT assay of normal human melanocytes treated with varying concentrations of IVMT-Rx-3. The assay demonstrates a lack of effect of IVMT-Rx-3 on cell proliferation in early passage primary normal human melanocytes (NHEM).

FIG. 2B is a histogram showing the results of a colony formation assay of normal human melanocytes treated with varying concentrations of IVMT-Rx-3. Two-hundred cells were plated on 60-mm dishes, treated with IVMT-Rx-3 at different doses. Every third day, culture media was replaced with fresh IVMT-Rx-3-containing media. After two weeks, colonies were stained, counted and graphs plotted.

FIG. 2C is a series of images showing colony formation assays of established human melanoma and patient-derived early passage human melanoma cells treated with IVMT-Rx-3.

FIG. 2D is a series of histograms showing the number of colonies/plate in the colony formation assays described in FIG. 2C. NS: Not statistically significant; Statistically significant p<0.05.

FIG. 2E is a series of histograms showing the number of colonies/plate in colony formation assays of patient-derived early passage human melanoma cells treated with IVMT-Rx-3. NS: Not statistically significant; Statistically significant p<0.05.

FIG. 3A is a series of photomicrographs taken at 10× magnification of established human melanoma cell lines (A-375, C8161.9, MeWo) and early passage patient-derived human melanoma cells (TPF-19-219, TPF-19-235, TPF-16-238) treated for 12 hours with DMSO vehicle or IVMT-Rx-3 at different doses (as indicated). Invasion was assayed using a modified Boyden chamber.

FIG. 3B is a series of histograms showing the average percent of invasion±standard deviation of the established human melanoma cell lines (A-375, C8161.9, and MeWo cells) in the experiments described in FIG. 3A. *: Statistical significance (p<0.05).

FIG. 3C is a series of histograms showing the average percent of invasion±standard deviation of the early passage patient-derived human melanoma cells (TPF-19-219, TPF-19-235, and TPF-16-238 cells) in the experiments described in FIG. 3A. *: Statistical significance (p<0.05).

FIG. 4A is an immunoblot of C8161.9 and A-375 cells treated for 12 hours with either DMSO vehicle or IVMT-Rx-3 at different doses (as indicated) and re-plated onto fibronectin-coated plates. After 30 minutes, cell lysates were immunoprecipitated and immunoblotted with the indicated antibodies. The immunoblots show treatment with IVMT-Rx-3 disrupted MDA-9/Syntenin: Src interactions.

FIG. 4B is a Western blot of C8161.9 and A-375 cells treated with indicated doses of IVMT-Rx-3 for 24 hours. Cell lysates were subjected to Western blotting analysis to determine the expression levels of various signaling proteins (as shown) associated with MDA-9/Syntenin: Src interactions. Western blotting densitometry and relative intensity of protein bands were calculated using Image J software. The values are provided.

FIG. 4C is a histogram showing the NF-κB transcriptional activity of A-375 melanoma cells treated with the indicated doses of IVMT-Rx-3. *: Statistical significance (p<0.05).

FIG. 4D is a histogram showing the NF-κB transcriptional activity of C8161.9 melanoma cells treated with the indicated doses of IVMT-Rx-3. *: Statistical significance (p<0.05).

FIG. 5A is a series of histograms showing the MMP-2 and MMP-9 mRNA transcriptional activity in established human melanoma cells (A-375, C8161.9, and MeWo cells) and patient-derived early passage human melanoma cells (TPF-19-219, TPF-19-235, and TPF-16-238 cells) treated with IVMT-Rx-3 at varying doses, as determined by qPCR. *: Statistical significance (p<0.05).

FIG. 5B is images of zymography assays of MMP-2 and MMP-9 activity in A-375, C8161.9 cells treated with IVMT-Rx-3 at varying doses, and histograms showing the calculated intensity from the zymography assays. *: Statistical significance (p<0.05).

FIG. 5C is a series of histograms showing the amount of IGFBP-2 secreted from A-375, C8161.9, TPF-19-219, TPF-19-235, and TPF-16-238 cells treated with varying concentrations of IVMT-Rx-3, as measured by ELISA.

FIG. 6A is a series of images of B16 lung metastases in C57BL/6 mice treated with DMSO vehicle, PDZ1i, or IVMT-Rx-3 (intraperitoneal injection of 30 mg/kg, 3×/week, 6 injections total). 1×10⁵ B16 cells were injected intravenously in C57BL/6 mice (n=7, per group) prior to treatment.

FIG. 6B is a series of histograms showing the number of nodules/lung, T cell populations (CD8⁺IFN-γ⁺) and myeloid-derived suppressor cells (MDSCs) in the lungs of the treatment groups described in FIG. 6A, as measured by flow cytometry. *: Statistical significance (p<0.05);**: Statistical significance (p<0.01);***: Statistical significance (p<0.001). ****: Statistical significance (p<0.0001).

FIG. 6C is a histogram showing the IGFBP-2 expression measured in blood plasma from animals (n=7) in the treatment groups described in FIG. 6A. ****: Statistical significance (p<0.0001).

FIG. 6D is a series of images of B16 lung metastases in C57BL/6 mice treated with DMSO vehicle, anti-PD-L1 antibody (intraperitoneal injection 100 μg, 3× per week, 2 week period), IVMT-Rx-3 (intraperitoneal injection 30 mg/kg, 3× per week, 6 injections in total), or IVMT-Rx-3+anti-PD-L1 antibody. Animals were sacrificed after 2 weeks and lungs were isolated.

FIG. 6E is a histogram showing the number of nodules/lung in the lungs of the treatment groups described in FIG. 6D. Different letters in different groups represent statistical differences.

FIG. 7A is graph showing Kaplan-Meier curves of the effect of IVMT-Rx-3, anti-PD-L1, and IVMT-Rx-3+anti-PD-L1 on survival versus control. The significance between groups was determined using the Log-Rank test.

FIG. 7B is a graphic showing the mechanism of action of IVMT-Rx-3. IVMT-Rx-3 deactivates NF-κB through disruption of MDA-9/Syntenin and cSRC interactions resulting in downregulation of invasion- and angiogenesis-associated gene(s) expression causing suppression of tumor cell dissemination. IVMT-Rx-3 also inhibitors NF-κB dependent inflammatory cytokine expression thereby enhancing anti-tumor immunity and preventing melanoma cell growth in the metastatic niche.

FIG. 8A is an HPLC trace for IVMT-Rx-3.

FIG. 8B is an HPLC trace for IVMT-Rx-3.

FIG. 8C is a QC LCMS trace and mass spectrum for IVMT-Rx-3.

FIG. 8D is a QC LCMS trace and mass spectrum for IVMT-Rx-3.

FIG. 8E is a ¹H NMR spectrum for IVMT-Rx-3.

FIG. 8F is a ¹H NMR spectrum for IVMT-Rx-3.

FIG. 9A shows NMR titration ITC data that reveals two inflection points, one tighter and the second weaker, with an overall dissociation constant of the complex, Kd<1 μM if fitted to a single binding event. NMR titration provides similar values and mapping studies confirm that the compound induces chemical shifts in both domains.

FIG. 9B. shows [¹⁵N, ¹H]-HSQC MDA-9/Syntenin spectra (20 μM) in presence of 40 UM IVMT-Rx-3.

FIG. 9C shows [¹³C, ¹H]-HSQC of ¹³C-Met-labeled-MDA-9/Syntenin spectra in absence (20 μM) and in presence of 40 μM IVMT-Rx-3; the binding site residues Met201, located in PDZ2 domain, and Met188, located in PDZ1 domain, are both clearly perturbed by the binding of bi-dentate IVMT-Rx-3.

FIG. 10A is a series of Western blots showing silencing of MDA-9 in patient derived melanoma cell lines (TPF-19-219, TPF-19-235, and TPF-16-238 cells) using Ad.5/3-shmda-9.

FIG. 10B is a series of images showing invasion assays of TPF-19-219, TPF-19-235, and TPF-16-238 cells with silencing of MDA-9 compared to control, indicating a loss of MDA-9 and invasion ability in patient derived melanoma cell lines.

FIG. 10C is a series of histograms showing results of the invasion assays described in FIG. 10B, indicating a loss of MDA-9 and invasion ability in patient derived melanoma cell lines. ****: Statistical significance (p<0.0001).

FIG. 11A is a series of photomicrographs taken at 10× magnification showing invasion assays of A-375 cells treated with DMSO vehicle, 25 μM PDZ1i, or 25 μM IVMT-Rx-3, as well as histograms showing quantification of the average percent invasion of three independent experiments±standard deviation. *: Statistical significance (p<0.05).

FIG. 11B is a series of photomicrographs taken at 10× magnification showing invasion assays of C8161.9 cells treated with DMSO vehicle, 25 μM PDZ1i, or 25 μM IVMT-Rx-3 in C8161.9 melanoma cells, as well as histograms showing quantification of the average percent invasion of three independent experiments±standard deviation. *: Statistical significance (p<0.05).

FIG. 12 is a Western blot of different signaling molecules as indicated for indicated cell lines treated with IVMT-Rx-3 at varying concentrations. The Western blot shows inactivation of various signaling pathways (as shown) associated with MDA-9/Syntenin: Src interactions across various melanoma cell lines (as shown).

FIG. 13A is a series of photomicrographs of B16 melanoma cells treated with IVMT-Rx-3 at varying concentrations, as well as a histogram showing quantification of the average percentage (of control) of invaded cells. Invasion was assayed using a Boyden chamber. *: Statistical significance (p<0.05).

FIG. 13B is a series of Western blots of indicated signaling molecules in the cells treated as described in FIG. 13A, indicating downregulation of Src and downstream P38 in IVMT-Rx-3-treated cells.

FIG. 13C is a series of histograms showing the MMP-2 and MMP-9 mRNA fold change in the cells treated as described in FIG. 13A, as determined by qPCR. *: Statistical significance (p<0.05).

FIG. 14 is a series of histograms showing the IL-6, IL-10, and IL-16 mRNA fold change in RNA lysates prepared from A-375, C8161.9, and B16 cells treated with IVMT-Rx-3 at varying concentrations as indicated.

FIG. 15 is a histogram showing the comparative effect of DMSO and IVMT-Rx-3 on Treg populations, as measured by flow cytometry. 5×10⁵ B16 cells were injected intravenously in C57BL/6 mice, and the mice were treated with DMSO control or IVMT-Rx-3 (intraperitoneal injection 30 mg/kg, 3× in a week, 6 injections in total). MDSC (Cd11⁺Gr1⁺) and Treg populations (CD4⁺CD25⁺Foxp3⁻) in the lungs were measured by flow cytometry.

### DETAILED DESCRIPTION

The present disclosure provides, in part, compounds that are useful for treating cancer (e.g., melanoma). In some embodiments, the compounds disclosed herein bind both PDZ domains of MDA-9/Syntenin. The present disclosure also provides pharmaceutical compositions comprising a compound disclosed herein (e.g., a compound of formula (I) or formula (Ia)) and one or more pharmaceutically acceptable excipients. Further provided herein are methods of treating cancer (e.g., melanoma) comprising administering to a subject in need thereof an effective amount of a compound or pharmaceutical composition disclosed herein.

Genome-wide gene expression analysis and animal modeling indicate that melanoma differentiation associated gene-9 (mda-9, Syntenin, Syndecan binding protein, referred to as MDA-9/Syntenin) positively regulates melanoma metastasis. The MDA-9/Syntenin protein contains two tandem PDZ domains serving as a nexus for interactions with multiple proteins that initiate transcription of metastasis-associated genes. Although targeting either PDZ domain abrogates signaling and pro-metastatic phenotypes, the integrity of both domains is critical for full biological function. Fragment-based drug discovery and NMR identified PDZ1i, an inhibitor of the PDZ1 domain that effectively blocks cancer invasion in vitro and in vivo in multiple experimental animal models. To maximize disruption of MDA-9/Syntenin signaling an inhibitor has now been developed that simultaneously binds and blocks activity of both PDZ domains. PDZ1i was joined to the second PDZ binding peptide (TNYYFV) with a PEG linker, resulting in PDZ1/2i (IVMT-Rx-3) that engages both PDZ domains of MDA-9/Syntenin. IVMT-Rx-3 blocks MDA-9/Syntenin interaction with Src, reduces NF-κB activation, and inhibits MMP-2/MMP-9 expression, culminating in repression of melanoma metastasis. The in vivo anti-metastatic properties of IVMT-Rx-3 are enhanced when combined with an immune-checkpoint inhibitor. Collectively, the present disclosure supports the feasibility of engineering MDA-9 dual-PDZ inhibitors with enhanced anti-metastatic activities and applications of IVMT-Rx-3 for developing novel therapeutic strategies effectively targeting melanoma and in principle, a broad spectrum of human cancers that also overexpress MDA-9/Syntenin.

Melanoma differentiation associated gene-9 (mda-9), also known as Syntenin-1 or Syndecan Binding Protein (SDCBP) (mda-9/syntenin) was initially cloned from terminally differentiating human melanoma cells (4,5) and shown to positively regulate melanoma progression and metastasis (6-9). In addition, using the dataset GSE3189, Bacolod et al. (10) reported differential expression of MDA-9/Syntenin in malignant melanoma in comparison with normal skin. When considering metastatic samples from different cancers, MDA-9/Syntenin expression was robust and ranked first among six carcinomas tested from TCGA datasets (10). Along with genetic and other molecular biological-based strategies using gain- and loss-of-function, the feasibility of this protein as a putative therapeutic molecular target has been pre-clinically validated in multiple independent research laboratories (11-15). Expression at relevant developmental stages (16) and contributing roles in several important physiological processes, such as exosome biogenesis, anti-viral activities, immunoglobulin regulation and pre-synaptic synapses, might be considered a red flag and signal safety concerns (reviewed by Das et al. (17)). However, the lack of lethality following global knockout of mda-9/syntenin in animals supports the possibility that targeting gene/protein might not be detrimental or toxic (18,19), which has been supported by further work (20-26).

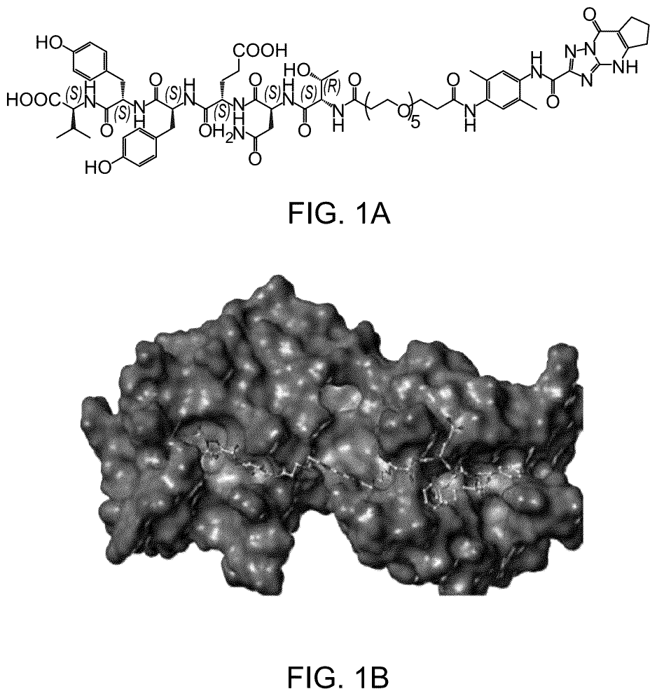

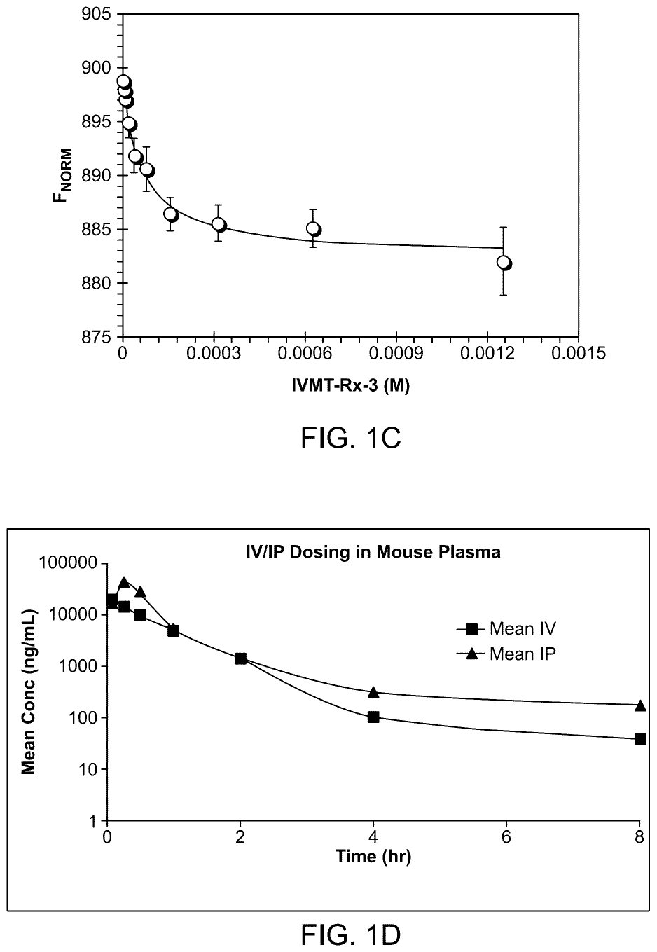

Structurally, MDA-9/Syntenin has two tandem PDZ domains (PDZ1 and PDZ2) that are both essential for metastasis (8). A novel MDA-9/Syntenin PDZ1-targeted molecule (PDZ1i) was previously developed to inhibit these activities (20-23), which binds to the first PDZ domain (PDZ1) and the interface between the first and second domain of PDZ1 and PDZ2, the interdomain. PDZ1i is described in U.S. Pat. No. 11,008,325, the entire contents of which is incorporated by reference herein. This interaction with PDZ1i occurs without appreciable binding to the second PDZ2 domain of MDA-9/Syntenin or PDZ domains in other sequence-similar proteins, confirming selectivity for MDA-9/Syntenin (20). Since both PDZ domains may be important in defining total activities of this molecule, efforts were undertaken to design molecules capable of binding to both PDZ domains to maximize disruption of MDA-9/Syntenin-mediated protein-protein interactions thereby inhibiting downstream signaling. Additionally, inhibitors of both domains might have expanded activity vs. inhibition of a single domain. Disclosed herein is the linking of the small molecule PDZ1i to a PDZ2 binding peptide (TNEYYF) using a PEG-linker to generate IVMT-Rx-3 (FIGS. 1A-1C; docking model, chemical structure and binding assay), that impairs the function of MDA-9/Syntenin at many biological levels, including in vitro and in vivo transformation-related phenotypes, without provoking toxicity in early passage normal human melanocytes or pre-clinical animal models.

While immunotherapy using check-point-based inhibitors has accomplished promising therapeutic outcomes in patients with melanoma and has been approved for use in melanoma by the US Food & Drug Administration, only a subset of melanoma patients respond favorably (27). However, accumulating evidence indicates that a combination of immunotherapy with other modalities such as targeted molecular therapies promote synergy thereby producing objective responses above what is attained with either agent alone (28). Accordingly, as described herein, the efficacy of the combination of a check-point inhibitor (anti-PD-L1 Ab), and IVMT-Rx-3 in controlling both cellular metastatic competence and tumor growth within the metastatic site was tested (e.g., lung).

### DEFINITIONS

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention. The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

As used herein, the term “alkyl” refers to a radical of a straight-chain or branched saturated hydrocarbon group, e.g., having 1 to 6 carbon atoms (“C₁₋₆ alkyl”). Examples of C₁₋₆ alkyl groups include methyl, ethyl, propyl, isopropyl, butyl, isobutyl, pentyl, hexyl, and the like

As used herein, the term “halo” refers to an atom selected from fluorine (fluoro, —F), chlorine (chloro, —Cl), bromine (bromo, —Br), and iodine (iodo, —I). In certain embodiments, the halo group is fluoro or chloro.

As used herein, the term “non-cleavable linker” refers to a linker that does not cleave in response to a chemical trigger (e.g., low pH, degradation by glutathione, etc.).

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The terms “a” or “an,” as used in herein means one or more.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., microRNA) may be detected by standard methods well known in the art. See, Sambrook et al., 1989 Molecular Cloning: A Laboratory Manual, 18.1-18.88.

“Patient” or “subject in need thereof” refers to a living organism suffering from or prone to a disease or condition that can be treated by administration of a compound, composition, or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “treating,” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In embodiments, “treating” refers to treatment of cancer.

In some embodiments, treatment or treating includes (1) inhibiting a disease in a subject or patient experiencing or displaying the pathology or symptomatology of cancer (e.g., arresting further development of the pathology and/or symptomatology), (2) ameliorating a disease in a subject or patient that is experiencing or displaying the pathology or symptomatology of cancer (e.g., reversing the pathology and/or symptomatology), and/or (3) effecting any measurable decrease in a disease in a subject or patient that is experiencing or displaying the pathology or symptomatology of cancer. In embodiments, the subject treated as described herein may also fully recover from cancer and may become cancer-free as a result of the present methods.

In embodiments, treating cancer refers to at least ameliorating and/or decreasing and/or eradicating aspects of the disease such as the following: the size of a tumor may be lessened and/or the tumor may be completely destroyed; remnants of a tumor (e.g. after surgery) may be lessened and/or destroyed; the growth of a tumor may be prevented and/or the growth rate may be slowed; the metastatic potential of a tumor may be decreased or eliminated; cancer cells may be sensitized to radiation therapy, etc. For example, when cancer cells are exposed to a compound or drug described herein prior to, during or after radiation therapy, they are more susceptible to killing by radiation, e.g. at least about 25% more of the cancer cells die without dividing, and typically at least about 50, 75 or even 100% of the cells die without dividing, compared to the number that die when exposed to radiation alone. In addition, in embodiments, select changes which typically occur in cancer cells when exposed to radiation are decreased or eliminated when a compound or drug described herein is administered to a subject receiving radiotherapy. For example, cancer cells frequently exhibit an increased ability to grow, divide, and/or metastasize after radiation therapy, and administration of the present drugs (e.g., compound described herein (e.g., a compound of formula (I) or formula (Ia))) attenuates or eliminates this ability. In embodiments, the treatment of cancer metastasis includes the treatment of at least one of invasion, migration, and angiogenesis.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” For example, for a given parameter, an effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, an effective amount can have at least a 1.2-fold, 1.5-fold, 2-fold, 5-fold, or more effect over a control.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The compositions described herein can be used in combination with one another, with other active agents known to be useful in treating a cancer such as anti-cancer agents.

### Compounds

The present disclosure provides, in part, compounds that are useful for treating cancer (e.g., melanoma).

In one aspect, provided herein is a compound of formula (I),

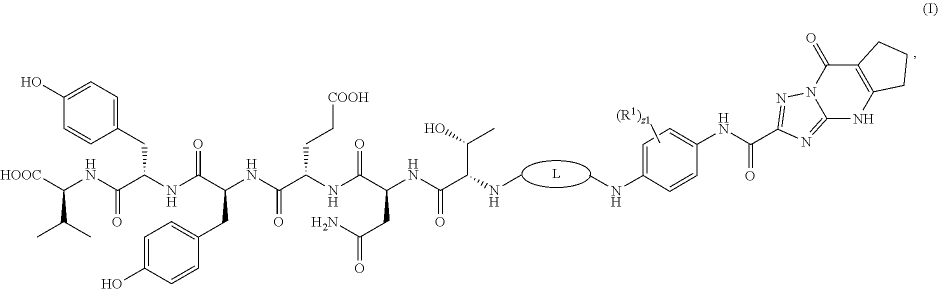

or a pharmaceutically acceptable salt thereof, wherein
L is a non-cleavable linker
each R¹ is independently selected from halo, —OH, and C₁₋₆ alkyl; and
z1 is an integer from 0 to 3.

In some embodiments, each R¹ is independently halo or C₁₋₆ alkyl. In some embodiments, each R¹ is C₁₋₆ alkyl. In some embodiments, each R¹ is independently methyl or fluoro. In some embodiments, each R¹ is methyl.

In some embodiments, z1 is 1. In some embodiments, z1 is 2. In some embodiments, z1 is 3.

In some embodiments, the compound is a compound of formula (Ia),

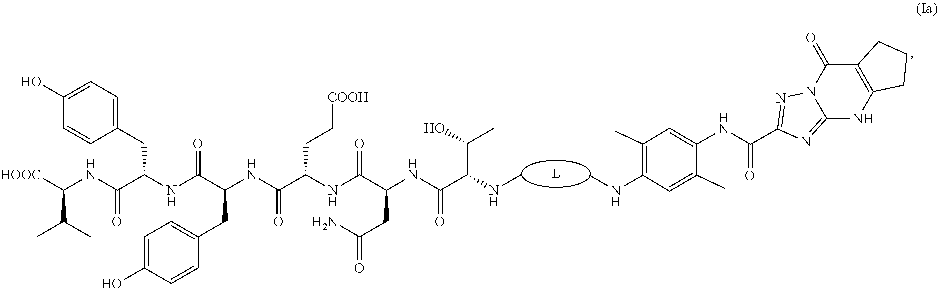 [SMILES: CN[C@H](C(=O)N[C@@H](CC(N)=O)C(=O)N[C@@H](CCC(=O)O)C(=O)N[C@@H](Cc1ccc(O)cc1)C(=O)N[C@@H](Cc1ccc(O)cc1)C(=O)N[C@H](C(=O)O)C(C)C)[C@@H](C)O ; CNc1cc(C)c(NC(=O)c2nc3[nH]c4c(c(=O)n3n2)CCC4)cc1C]

or a pharmaceutically acceptable salt thereof.

In some embodiments, L is at least 15 Angstroms in length. In some embodiments, L is at least 20 Angstroms in length. In some embodiments, L is 15-25 Angstroms in length.

In some embodiments, L is

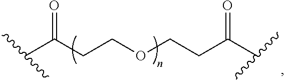

wherein n is an integer from 5 to 10. In some embodiments, n is an integer from 5 to 7. In certain embodiments, n is 5. In certain embodiments, n is 6. In certain embodiments, n is 7. In certain embodiments, n is 8. In certain embodiments, n is 9. In certain embodiments, n is 10.

In another aspect, provided herein is a compound represented by the formula

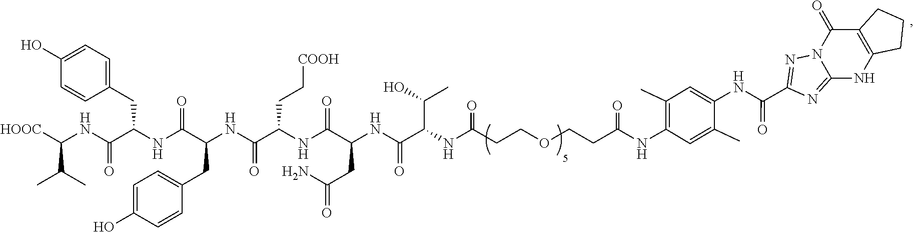

or a pharmaceutically acceptable salt thereof.

In another aspect, provided herein is a compound represented by the formula

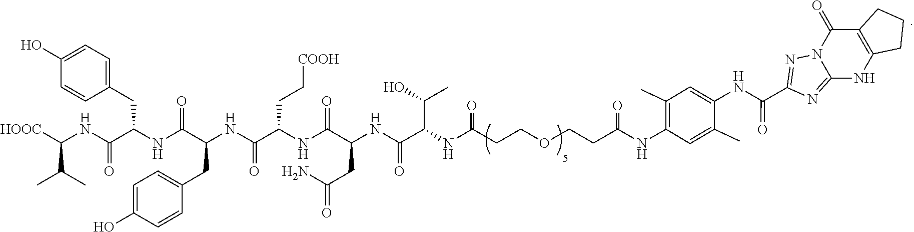

In another aspect, provided herein is a pharmaceutically acceptable salt of a compound represented by the formula

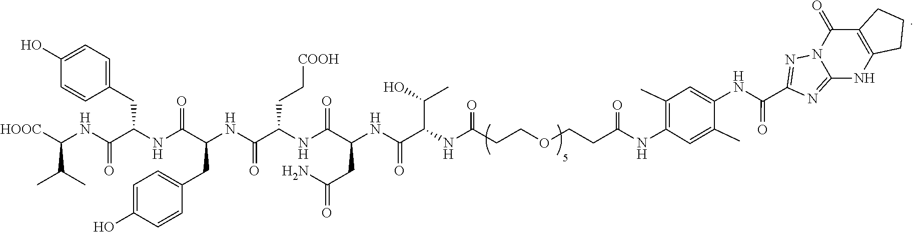

### Pharmaceutical Compositions

The present disclosure provides, in part, pharmaceutical compositions comprising a compound disclosed herein (e.g., a compound of formula (I) or formula (Ia)) and one or more pharmaceutically acceptable excipients.

The compounds described herein are generally delivered (administered) as a pharmaceutical composition. Such pharmaceutical compositions generally include at least one of the disclosed compounds (e.g., in pure form or salt), and more than one (a plurality) of different compounds (e.g. 2 or more such as 2, 3, 4, 5, 6, 7, 8, 9, 10 or more) may be included in a single formulation. Accordingly, the present invention encompasses such formulations and compositions. The compositions generally include one or more substantially purified compounds as described herein, and a pharmacologically suitable (physiologically compatible) excipient, which may be aqueous or oil-based. In some embodiments, such compositions are prepared as liquid solutions or suspensions. In other embodiments, the compositions are prepared in solid forms such as tablets, pills, powders and the like. Solid forms suitable for solution, dissolution or suspension in liquids prior to administration are also contemplated (e.g. lyophilized forms of the compounds), as are emulsified preparations. In some embodiments, the liquid formulations are aqueous or oil-based suspensions or solutions. In some embodiments, the active ingredients are mixed with excipients which are pharmaceutically acceptable and compatible with the active ingredients, e.g. pharmaceutically acceptable salts. Suitable excipients include, for example, water, saline, dextrose, glycerol, cyclodextrin, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added. The composition of the present invention may contain any such additional ingredients so as to provide the composition in a form suitable for administration. The final amount of compound in the formulations varies, but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which serve as pharmaceutically acceptable excipients include, but are not limited to, ion exchangers, alumina, aluminum stearate, lecithin, serum proteins (such as human serum albumin), buffer substances (such as Tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds (e.g., compounds described herein (e.g., compounds of formula (I) or formula (Ia))). These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

### Administration

In some embodiments, the formulation is administered in vivo by any suitable route including but not limited to: inoculation or injection (e.g. intravenous, intraperitoneal, intramuscular, subcutaneous, intra-aural, intraarticular, intramammary, intracranial, and the like), topical application (e.g. on any suitable skin or membrane surface), by absorption through epithelial or mucocutaneous linings (e.g., nasal, oral, vaginal, rectal, gastrointestinal mucosa, etc.) and the like. In embodiments, the compounds may be incorporated into implantable delivery means, e.g. drug permeated wafers, etc. Timed (sustained, extended, controlled) release formulations, e.g. in the form of pills, tablets, capsules, etc. are also contemplated, including various diffusion systems such as reservoir and matrix devices; osmotic, ion exchange, floating, bio-adhesive, and depot systems, etc. In some embodiments, the mode of administration is intranasal, orally or parenteral, by intravenous, intraperitoneal, intramuscular, topical or subcutaneous routes, and usually is by intravenous injection.

In embodiments, administration is carried out in a coordinated manner at time intervals which are spaced apart by minutes, hours, days or weeks, etc.

### Methods of Treatment/Medical Uses

The present disclosure provides, in part, methods of treating cancer (e.g., melanoma) or preventing cancer metastasis (e.g., melanoma metastasis) comprising administering to a subject in need thereof an effective amount of a compound or pharmaceutical composition disclosed herein.

In one aspect, provided herein is a method of treating cancer in a subject in need thereof, the method comprising administering to the subject an effective amount of a compound or pharmaceutical composition disclosed herein.

In another aspect, provided herein is a method of treating cancer in a subject in need thereof, the method comprising administering to the subject an effective amount a compound or pharmaceutical composition disclosed herein, and an effective amount of an α-PD-L1 checkpoint inhibitor. In some embodiments, the α-PD-L1 checkpoint inhibitor is selected from the group consisting of atezolizumab, avelumab, durvalumab, KN035, cosibelimab, AUNP12, CA-170, and BMS-986189. In some embodiments, the α-PD-L1 checkpoint inhibitor is selected from the group consisting of atezolizumab, avelumab, and durvalumab.

In some embodiments, the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma. In some embodiments, treating the cancer comprises preventing or slowing the development of metastasis of the cancer. In some embodiments, the cancer is melanoma. In some embodiments, the melanoma is metastatic melanoma. In some embodiments, treating melanoma comprises preventing or slowing the development of melanoma lung metastases. In some embodiments, the melanoma comprises a p53 mutation. In some embodiments, the melanoma does not comprise a p53 mutation.

In some embodiments, the treating comprises reducing nuclear factor kappa-light-chain-enhancer of activated B cells (NF-κB) signaling in the cancer by administering to the subject a compound or pharmaceutical composition disclosed herein. In some embodiments, the treating comprises reducing matrix metalloproteinase 2 (MMP-2) and/or matrix metalloproteinase 9 (MMP-9) expression in the cancer by administering to the subject a compound or pharmaceutical composition disclosed herein. In some embodiments, the treating comprises reducing insulin growth factor binding protein-2 (IGFBP-2) expression in the cancer by administering to the subject a compound or pharmaceutical composition disclosed herein. In some embodiments, the treating comprises reducing interleukin-6, interleukin-10, and/or interleukin-16 expression in the cancer by administering to the subject a compound or pharmaceutical composition disclosed herein. In some embodiments, the treating comprises increasing IFN-γ-producing CD8⁺ T cell populations in the subject by administering to the subject a compound or pharmaceutical composition disclosed herein. In some embodiments, the treating comprises decreasing myeloid-derived suppressor cell (MDSC) populations in the subject by administering to the subject a compound or pharmaceutical composition disclosed herein.

In another aspect, provided herein is a method of preventing or slowing the development of cancer metastasis in a subject with cancer, the method comprising administering to the subject an effective amount of a compound or pharmaceutical composition disclosed herein.

In another aspect, provided herein is a method of preventing or slowing the development of cancer metastasis in a subject with cancer, the method comprising administering to the subject an effective amount of a compound or pharmaceutical composition disclosed herein, and an effective amount of an α-PD-L1 checkpoint inhibitor. In some embodiments, the α-PD-L1 checkpoint inhibitor is selected from the group consisting of atezolizumab, avelumab, durvalumab, KN035, cosibelimab, AUNP12, CA-170, and BMS-986189. In some embodiments, the α-PD-L1 checkpoint inhibitor is selected from the group consisting of atezolizumab, avelumab, and durvalumab.

In some embodiments, the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma. In some embodiments, treating the cancer comprises preventing or slowing the development of metastasis of the cancer. In some embodiments, the cancer is melanoma. In some embodiments, the melanoma is metastatic melanoma. In some embodiments, treating melanoma comprises preventing or slowing the development of melanoma lung metastases. In some embodiments, the melanoma comprises a p53 mutation. In some embodiments, the melanoma does not comprise a p53 mutation.

In some embodiments, the preventing or slowing the development of the cancer comprises reducing nuclear factor kappa-light-chain-enhancer of activated B cells (NF-κB) signaling in the cancer by administering to the subject a compound or pharmaceutical composition disclosed herein. In some embodiments, the preventing or slowing the development of the cancer comprises reducing matrix metalloproteinase 2 (MMP-2) and/or matrix metalloproteinase 9 (MMP-9) expression in the cancer by administering to the subject a compound or pharmaceutical composition disclosed herein. In some embodiments, the preventing or slowing the development of the cancer comprises reducing insulin growth factor binding protein-2 (IGFBP-2) expression in the cancer by administering to the subject a compound or pharmaceutical composition disclosed herein. In some embodiments, the preventing or slowing the development of the cancer comprises reducing interleukin-6, interleukin-10, and/or interleukin-16 expression in the cancer by administering to the subject a compound or pharmaceutical composition disclosed herein. In some embodiments, the preventing or slowing the development of the cancer comprises increasing IFN-γ-producing CD8⁺ T cell populations in the subject by administering to the subject a compound or pharmaceutical composition disclosed herein. In some embodiments, the preventing or slowing the development of the cancer comprises decreasing myeloid-derived suppressor cell (MDSC) populations in the subject by administering to the subject a compound or pharmaceutical composition disclosed herein.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### CHEMICAL EXAMPLES

### Chemical Example 1—Synthesis of IVMT-Rx-3

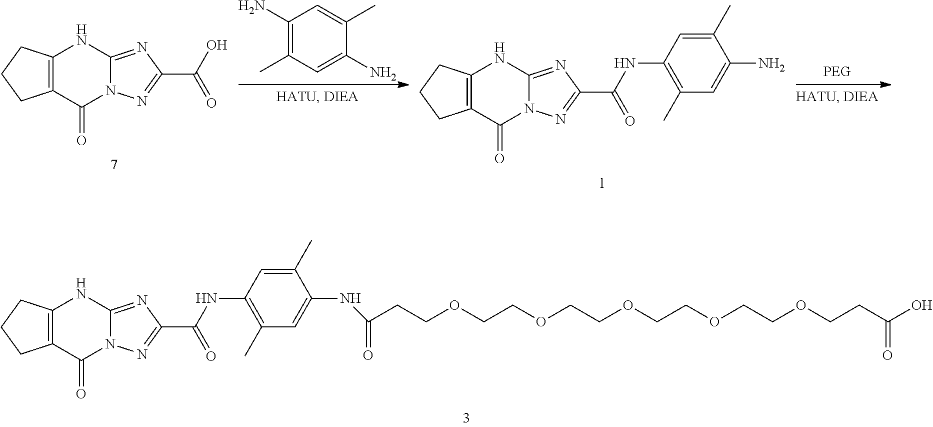

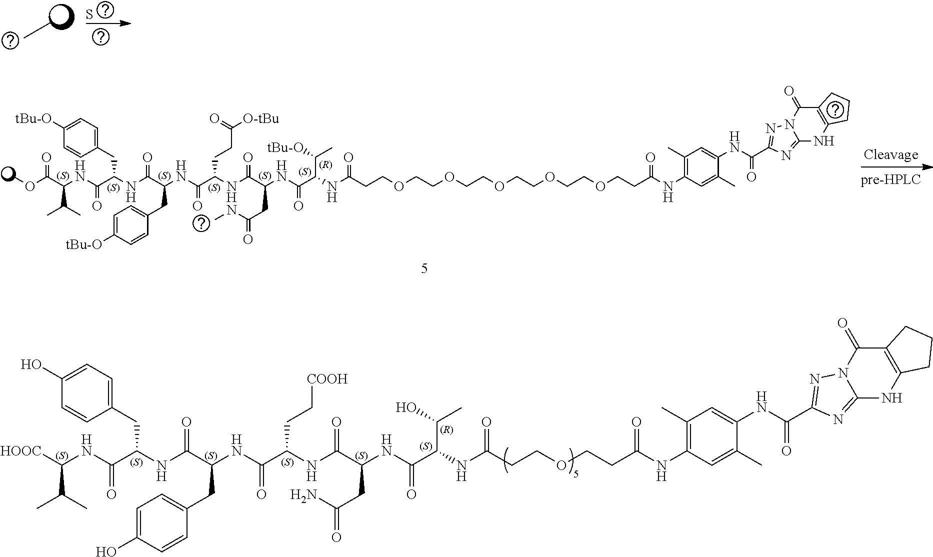

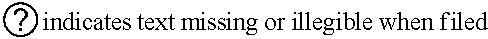

### Step 1: Synthesis of Compound 1

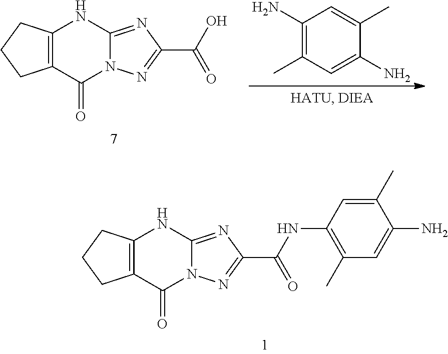

To a solution of compound 7 (20.0 g, 90.8 mmol, 1.00 eq) and 2,5-dimethylbenzene-1,4-diamine (13.6 g, 99.9 mmol, 1.10 eq) in DMF (200 mL) was added EDCI (34.8 g, 181 mmol, 2.00 eq), HOBt (24.5 g, 181 mmol, 2.00 eq) and TEA (27.5 g, 272 mmol, 37.9 mL, 3.00 eq) at 25 °C. The mixture was stirred at 25 °C for 16 h. LCMS (EW13407-12-P1A) showed that the compound 7 was consumed and the desired ms (Rt=0.482 min) was detected. The mixture was filtered, the filter cake was washed with DMF (200 mL) and dried to give white solid. LCMS showed that compound 1 (33.0 g, 98.3% yield, 91.6% purity) was obtained as a white solid. LC/MS: [M+H]⁺ 339.0

### Step 2: Procedure for Preparation of Peptide

The peptide was synthesized using standard Fmoc chemistry:
1) DCM was added to the vessel containing Fmoc-Rink Amide MBHA Resin (20 mmol, 17.1 g, 1.17 mmol/g) with N₂ bubbling for 30 minutes.
2) The vessel was drained and the resin washed with DMF 3 times.
3) 20% piperidine in DMF was added to the vessel and reacted for 30 minutes.
4) The vessel was drained and the resin washed with DMF 3 times.
5) Fmoc-amino acid solution was added and mixed for 30 second, followed by activation buffer, and the mixture was stirred with N₂ bubbling for about 1 hour.
6) Steps 2-5 were repeated for next amino acid couplings.

| # | Materials | Coupling reagents |
|---|---|---|
| 1 | Fmoc-Val-OH (3.00 eq) | HBTU (2.85 eq) and DIEA (6.00 eq) |
| 2 | Fmoc-Tyr(tBu)-OH (3.00 eq) | HBTU (2.85 eq) and DIEA (6.00 eq) |
| 3 | Fmoc-Tyr(tBu)-OH (3.00 eq) | HBTU (2.85 eq) and DIEA (6.00 eq) |
| 4 | Fmoc-Glu(OtBu)-OH 3.00 eq) | HBTU (2.85 eq) and DIEA (6.00 eq) |
| 5 | Fmoc-Asn(Trt)-OH 3.00 eq) | HBTU (2.85 eq) and DIEA (6.00 eq) |
| 6 | Fmoc-Thr(tBu)-OH 3.00 eq) | HBTU (2.85 eq) and DIEA (6.00 eq) |
| 7 | EW13407-13-P1 (1.50 eq) | HOBT (1.5 eq) and DIC(1.50 eq) |

20% piperidine in DMF was used for Fmoc deprotection for 30 min. The coupling reaction was monitored by ninhydrin test, and the resin was washed with DMF for 5 times.

### Step 3: Peptide Cleavage and Purification of Final Product

1) Cleavage buffer (90% TFA/3.3% TIS/3.3% Thioanisole/3.3% H₂O) was added to the flask containing the side chain protected peptide at room temperature and stirred for 2 hr.
2) The solvent was removed under vacuum to give the crude product
3) The crude peptide was purified by Prep-HPLC (A: 0.075% TFA in H₂O, B: 20% ACN in THF) and lyophilized to give the final product (5.20 g, 6.1% yield)

### Purification Conditions

Purification condition

| | |
|---|---|
| Dissolution condition | Dissolve in 20% ACN-10% dmf |
| Instrument | Gilson GX-281 |
| Mobile Phase | A: H₂O (0.075% TFA in H₂O) |
| | B: 20% ACN in THF |
| Gradient | 30 °C/12-42-60 min, t = 47 min ; 30 °C/ |
| | 30-60-20 min, t = 10.025 |
| Column | luna, c18, 10 um, 100A + Gemini, 5 um, |
| | c18, 110A |
| Flow Rate | 20 mL/Min |
| Wavelength | 220/254 nm |
| Oven Tem. | 0 °C |

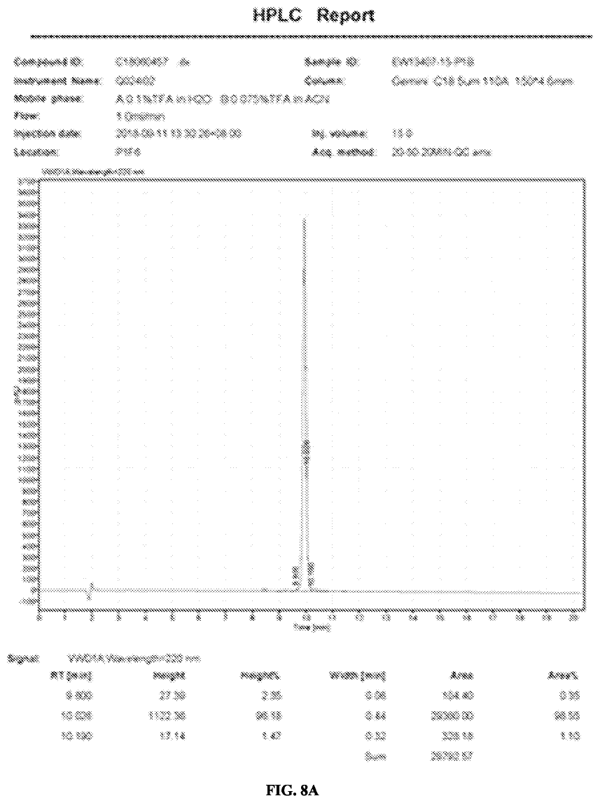

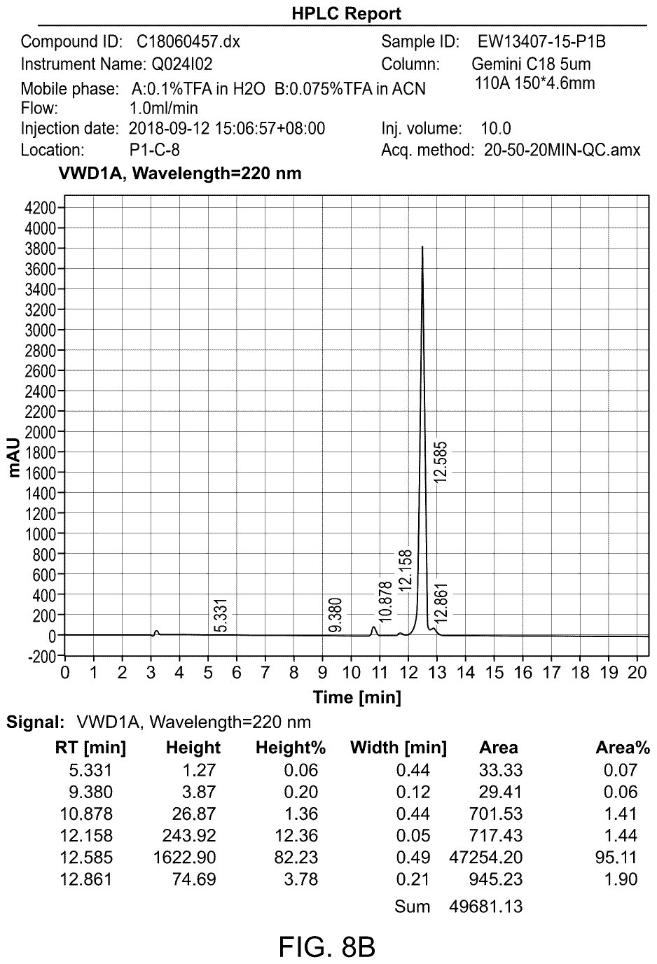

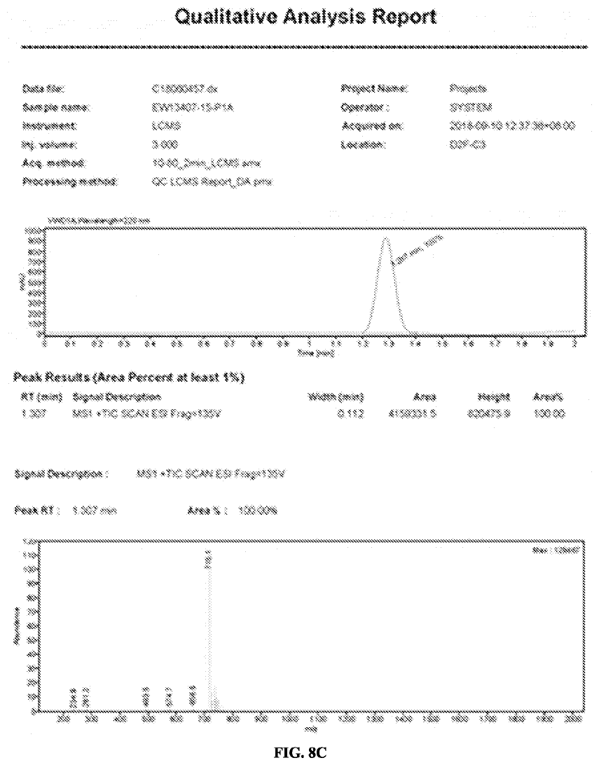

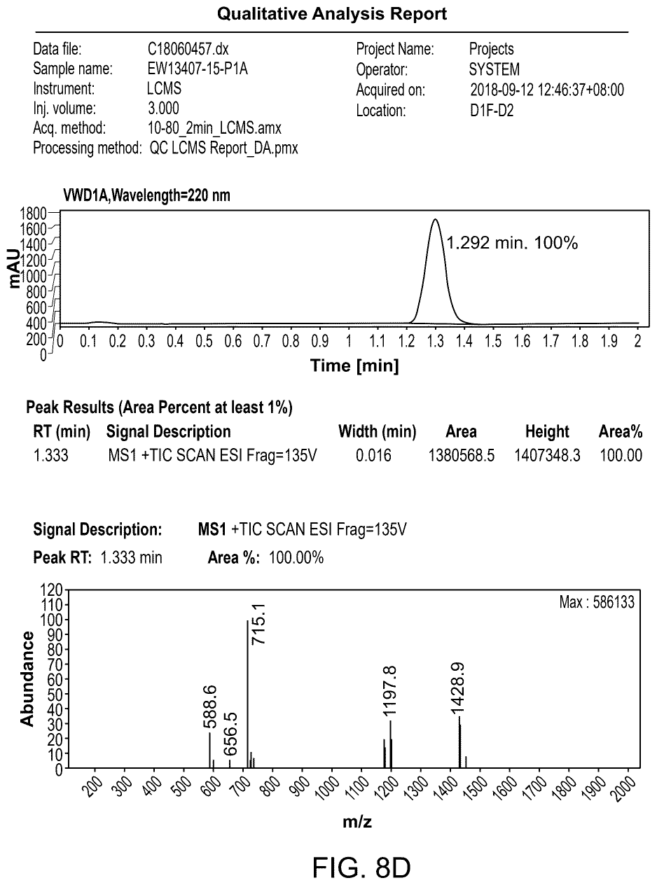

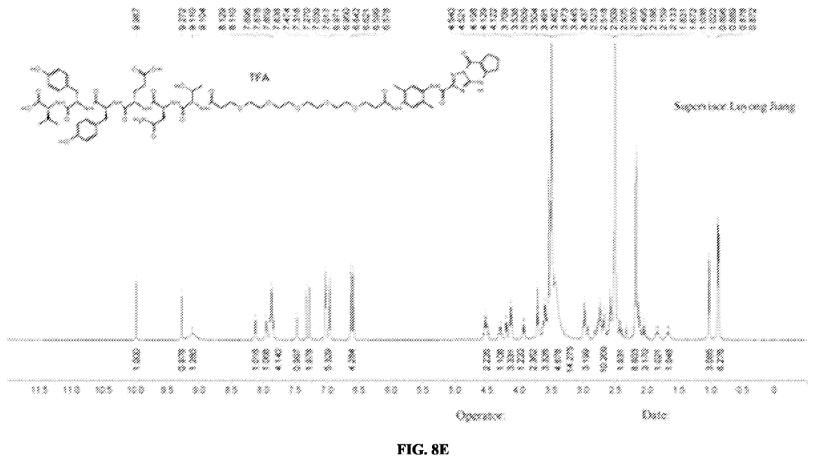

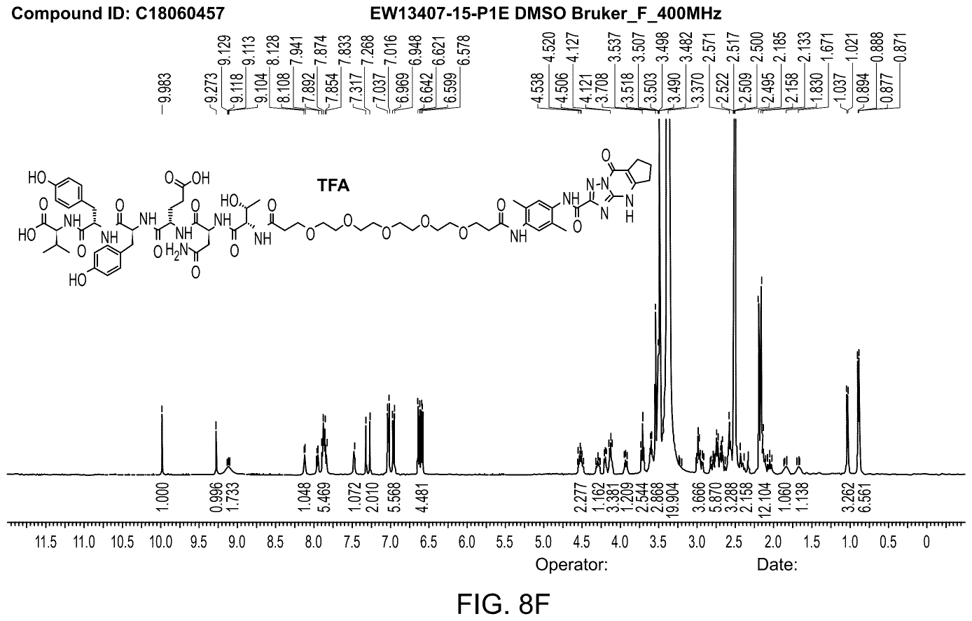

LC/MS: [M+H]⁺ 1428.9 Characterization details are provided as FIGS. 8A-8F.

### BIOLOGICAL EXAMPLES

### General Protocols

### Cell Lines and Reagents

Multiple melanoma cell lines of human origin were used in this study, which were maintained with appropriate media as recommended by the provider. C8161.9 and MeWo were gifts from Dr. Danny R. Welch (Kansas University Medical Center, Kansas City, KS) and Dr. Robert S. Kerbel (Sunnybrook Cancer Center, Toronto, Canada), respectively. RPMI-7951 and A-375 cells were purchased from the ATCC (Manassas, VA) and maintained according to their provider. The murine melanoma cell line B16 was provided by Dr. X.-Y. Wang (Virginia Commonwealth University, School of Medicine). Absence of mycoplasma during routine cell culture and animal experimentation was confirmed using a PCR-based mycoplasma detection kit (Millipore Sigma, SKU #MP0035 USA). Early passage primary human patient derived melanoma cultures were provided by Dr. John Kirkwood, University of Pittsburgh Cancer Center. The media used for the patient-derived melanoma cultures is RPMI-1640 w/L-glutamine that contains 10% heat inactivated FBS, 10 mM HEPES, 1×NEAA, 1 mM-L-glutamine, 1% Pen/Strep.

### Animals and In Vivo Experiments

All animal experiments were performed according to guidelines established by VCU IACUC. Immune competent mice (6-week-old male C57BL/6) were used in this study for determining the anti-metastatic efficiency of IVMT-Rx-3. Mice were purchased from Jackson Laboratories and after one-week in barrier facilities, experiments were performed. To establish lung metastases, murine B16 (1×10⁵ cells/0.1 ml PBS) were injected via tail vein in C57BL/6 mice (18). Test chemical (IVMT-Rx-3) was administrated at 30 mg/kg body weight three times a week I.P. for the first two weeks (six injections, C57BL/6 study). DMSO was used as control. Anti-PD-L1 antibody and control antibodies for therapeutic injections were purchased from Bio X Cell (West Lebanon, NH, USA).

1×10⁵ B16 cells were injected intravenously in C57BL/6 mice and treated with IVMT-Rx-3 (intraperitoneal injection 30 mg/kg, 3× in a week, 6 injections in total). 100 μg of Anti-PD-L1 antibody was injected through the I/P (intraperitoneal) route (3 times a week, over a 2-week period). A set of 20 animals were first injected with B16 cells (1×10⁵ cells/mouse) and randomly divided into four groups (n=5) for survival studies. As stated in the above-mentioned lung metastasis protocol, mice received 6 doses of control vehicle or IVMT-Rx-3 either alone or in combination with anti-PD-L1 over the first two weeks. Mice were maintained until they required euthanasia and Kaplan-Meier survival curves were generated GraphPad Prism software.

### Invasion Assays

Invasive phenotype was measured using a Boyden Chamber Assay as described previously (6,8,18). Briefly, 25,000 cells were plated on the upper chamber of the trans well in FBS free media and allowed to invade the Matrigel layer towards the bottom chamber supplemented with complete media. Twenty-four hr. after plating, the invaded cells were fixed and counted under a bright field microscope.

### Western Blotting Analysis

Western blotting was performed following a standard protocol as described previously (6,8,18,29). Antibodies used in this study were purchased from Cell Signaling Technology (Boston, MA): p-Src (Y416), total Src, matrix metalloproteinase-2 (MMP-2) and matrix metaloproteinase-9 (MMP-9), focal adhesion kinase (FAK), p IKKα/β (S176/180), total IKK, p P38 (T180/Y182) and total P38. MDA-9/Syntenin antibody was obtained from Abnova Inc. (Taiwan). β-actin (Sigma-Aldrich, St. Louis, MO) was used as an endogenous loading control. Fibronectin was purchased from EMD Millipore (Billerica, MA).

### Flow Cytometry

Single cells were isolated from lungs by digestion with collagenase D and DNAse I. Cells were stained with the corresponding antibodies and fluorescence signals were measured with a BD Fortessa flow cytometer (BD Biosciences, San Jose, CA). Data were analyzed further with FACs DIVA software (BD Biosciences, San Jose, CA) (18). The antibodies used in this study were CD3, Gr1, CD11b, IFN-γ, CD45, CD8 (Biolegend, San Diego, CA, USA). CD4 (BD Biosciences, San Jose, CA, USA), CD25, and FOXP3 (eBiosciences, CA, USA).

### Co-Immunoprecipitation

Co-immunoprecipitation studies were performed as described previously using immunoprecipitation kits with Dynabeads® Protein G (Life Technologies) (7,8). Briefly, 30 min. after plating cells on fibronectin-coated plates, protein lysates were prepared. 200 μg of total cellular protein was incubated with anti-IgG or anti-MDA-9 overnight to facilitate binding. After subsequent washing and elution from beads, Western blotting was performed with specific antibodies.

Real Time qPCR

Total cellular RNA was extracted using an RNAeasy kit (Qiagen, Germany) as described previously (8,9). All Taqman probes used in this study were obtained from Applied Biosystems (Applied Biosystems, Foster City, CA). Real time PCR was performed using an ABI ViiA7 fast real-time PCR system (Applied Biosystems, Foster City, CA).

NF-κB (p65) Transcription Factor Assays

NF-κB transcription factor assay kits were obtained from Cayman Chemical (Ann Arbor, MI, USA) (Cat #10007889). Nuclear extracts were prepared from cells using the Nuclear and Cytoplasmic Extraction kit (Thermo Fisher Scientific, USA). The samples were then added to pretreated wells for 1 hr. after which they were washed with a wash buffer provided in the kit. The NF-κB antibody was then added with a 1 hr. incubation and after washing the antibody, HRP-conjugated secondary antibody was added for 1 hr. Lastly, a developing solution followed by a stop solution was added. The wells were analyzed with a microplate reader for absorbance at 450 nm.

### Gelatin Zymography

Tumor cells were treated with different doses of IVMT-Rx-3 (10, 25, and 50 μM) for 24 hrs. The cells were then cultured in serum-free medium overnight. Conditioned media (serum free media) collected on the next day was quantified and equal amounts of protein were run in a 7.5% SDS-polyacrylamide gel electrophoresis containing 1.5 mg/mL gelatin. The gels were washed vigorously with shaking 3×in 2.5% Triton X-100 solution to remove the SDS. The gel was then placed in the developing buffer [50 mM/L Tris-HCl (pH 7.5), 0.05% NaN₃, 5 mM/L CaCl₂), and 1 μM/L ZnCl₂] 37 °C overnight. Lastly, the gels were stained with Amido black in 10% acetic acid followed by de-staining for 1 hr. MMP activities as observed through gelatinolysis were measured as clear zones of white strips against a blue background (21).

### ELISA Assays

IGFBP-2 levels in conditioned media were determined using an IGFBP-2 ELISA kit (R&D systems, USA) according to the manufacturer's instructions (9). Each sample was repeated three times, and represented graphically with GraphPad Prism software.

### In Vitro Pharmacokinetic Studies

These studies were conducted at the Sanford-Burnham Pharmacology Core facility for measurements of IVMT-Rx-3 in plasma after dosing.

### Binding Affinity Studies

Binding affinity was measured using microscale thermophoresis (MST) experiments performed on a Monolith NT. Automated instrument (NanoTemper Technologies GmbH, Munich, Germany). His-tagged MDA-9/Syntenin was first labeled with RED-tris-NTA (λEX-650 nm, λEM=670 nm; Cat #MO-LO18) according to the manufacturer's protocol. Briefly, 700 nM MDA-9 in 100 mM phosphate-buffered saline, pH 7.4, containing 0.05% Tween 20 (PBS-T) was added to an equal volume of 100 nM RED-tris-NTA in PBS-T and the mixture was incubated at room temperature for 30 min, centrifuged at 15,000 g for 10 min at 4 °C, and the supernatant containing the labeled protein was transferred to a new tube for use in affinity measurements. Following this, serial dilutions of IVMT-Rx-3 in PBS-T were prepared to which was added RED-tris-NTA labeled MDA-9/Syntenin (10 μL) with gentling mixing. The final protein concentration was 175 nM, while IVMT-Rx-3 concentrations ranged from 1.25 mM to 0.61 μM. After a 10-min incubation, the samples were loaded into premium capillary chips and thermophoretic measurements were performed (25 °C, 27% excitation power, medium MST power, 20 s laser on) to follow changes in normalized fluorescence (Fnorm) at different IVMT-Rx-3 concentrations (FIG. 1C). These changes were analyzed using the standard hyperbolic binding isotherm (SigmaPlot) to obtain the affinity of IVMT-Rx-3 for MDA-9/Syntenin. A minimum of five measurements were performed to calculate the affinity. The error in KD represents±S.E.

### Statistical Analysis

Unless otherwise stated, the in vitro experimental data were presented as the mean±S.D. of the values from three independent determinations. Statistical significance analysis was performed using the Student's t test and Wilcoxon Rank test in comparison with corresponding controls. Probability values<0.05 were considered statistically significant. For in vivo experiments, survival curves were analyzed using Cox proportional hazards survival regression using GraphPad Prism software.

### BIOLOGICAL EXAMPLES

### Biological Example 1—Characterization of IVMT-Rx-3

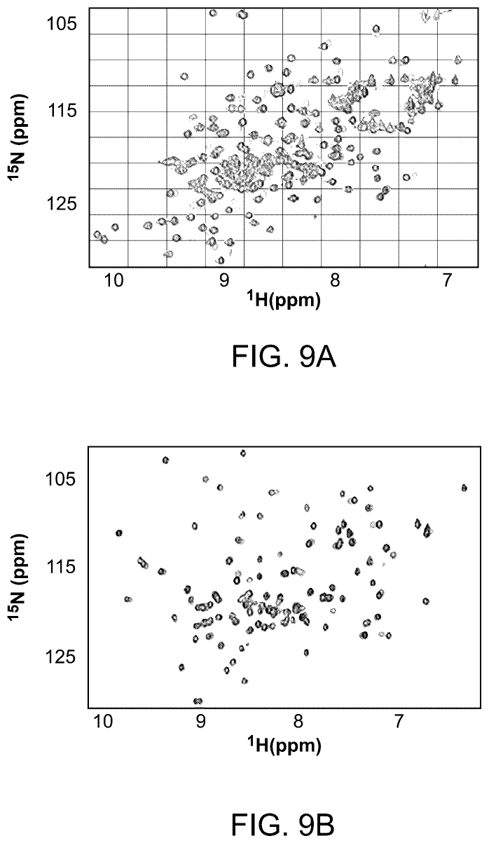

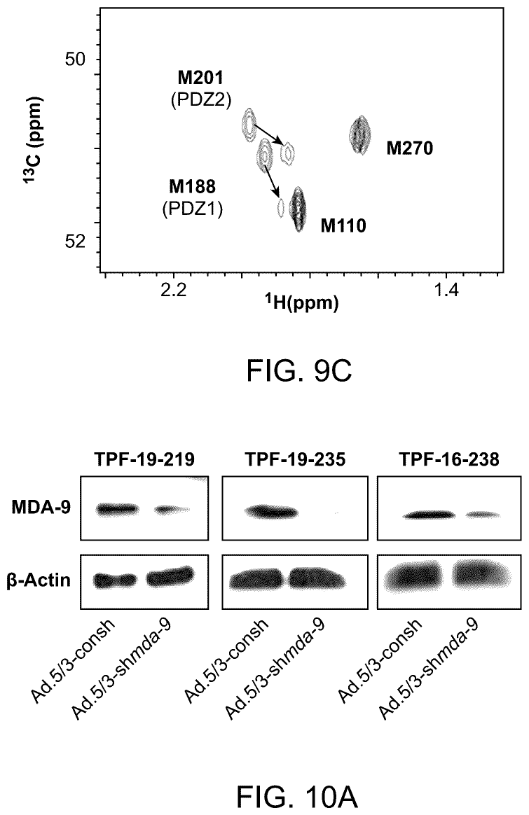

The chemical formula of IVMT-Rx-3 (PDZ1/2i) is provided as FIG. 1A, and synthesis schema is described above in Chemical Example 1. Docked structure of IVMT-Rx-3 in complex with MDA-9/Syntenin is provided as FIG. 1B. After confirming the synthesis product using different analytical methods, e.g., HPLC, LC/MS and NMR (FIGS. 8A-8F), the dissociation constant of IVMT-Rx-3-recombinant MDA-9/Syntenin complex was measured to be 63+/−11 μM using microscale thermophoresis (MST) analysis (FIG. 1C). In order to assess if this hybrid chimeric molecule (small molecule linked to a peptide) was suitable for cellular and in vivo assays, initial PK studies in mice were performed with IVMT-Rx-3 administered both IV (5.0 mg/kg) and IP (50.0 mg/kg), and compound concentration in serum was measured at the indicated times (FIG. 1D). IVMT-Rx-3 was long-lived in vivo, despite the peptide content, and it possessed a 10% bioavailability when given IP. IVMT-Rx-3 did not bind appreciably to other PDZ domains such as the PDZ tandem protein Harmonin (36% identity to PDZ2 of MDA-9/Syntenin and 31% to PDZ1 of MDA-9/Syntenin) or X11/mint scaffold protein (33% identity with PDZ1 shown) (FIGS. 9A-9C).

### Biological Example 2—Toxicity and Invasion Assays of Normal Human Melanocytes and Melanoma Cells Treated with IVMT-Rx-3

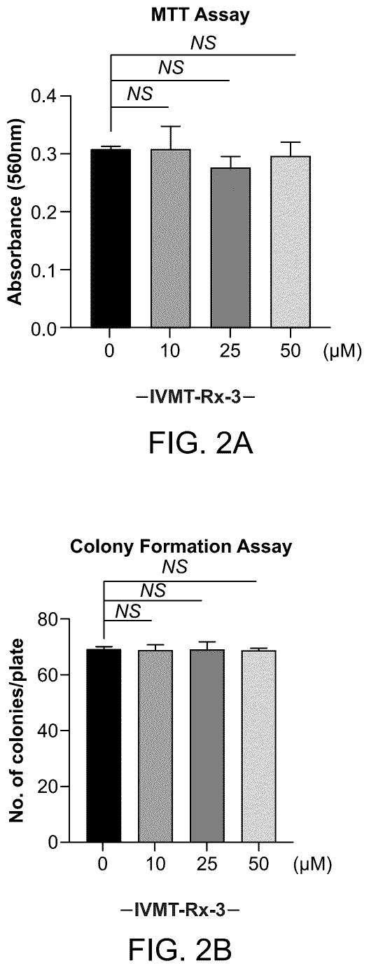

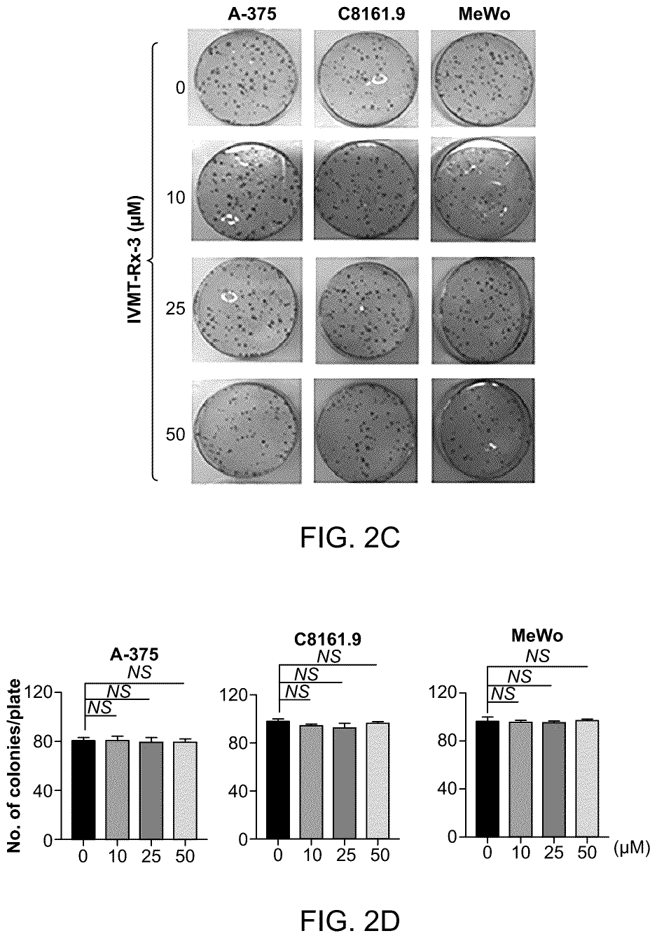

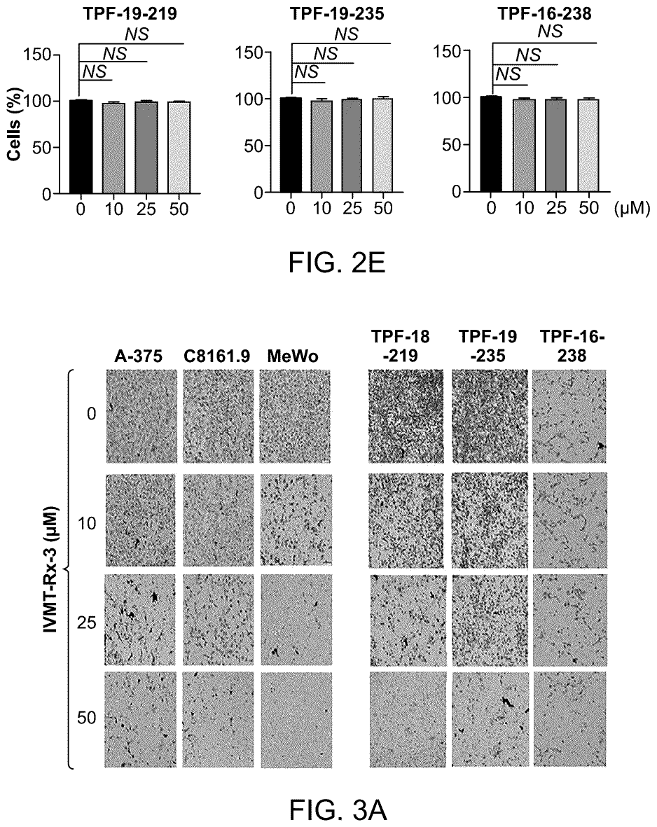

The effect of IVMT-Rx-3 on primary normal human epidermal melanocyte cell growth and survival was determined after short term (24 hr., growth assays) (FIG. 2A) and long-term exposure (3-week, clonal assays) (FIG. 2B). No significant toxicity was evident in primary melanocytes treated with 50 μM IVMT-Rx-3. Similarly, treatment with IVMT-Rx-3 (10 to 50 μM), did not inhibit proliferation or long-term survival in a wide range of human melanoma cells including both established (FIG. 2C and FIG. 2D) and early passage patient-derived cells (FIG. 2E). These finding indicate that, as observed previously with PDZ1i (20-22) in other cancer contexts, targeting MDA-9/Syntenin, in this case both PDZ domains, is not detrimental for either non-transformed normal or transformed cells.

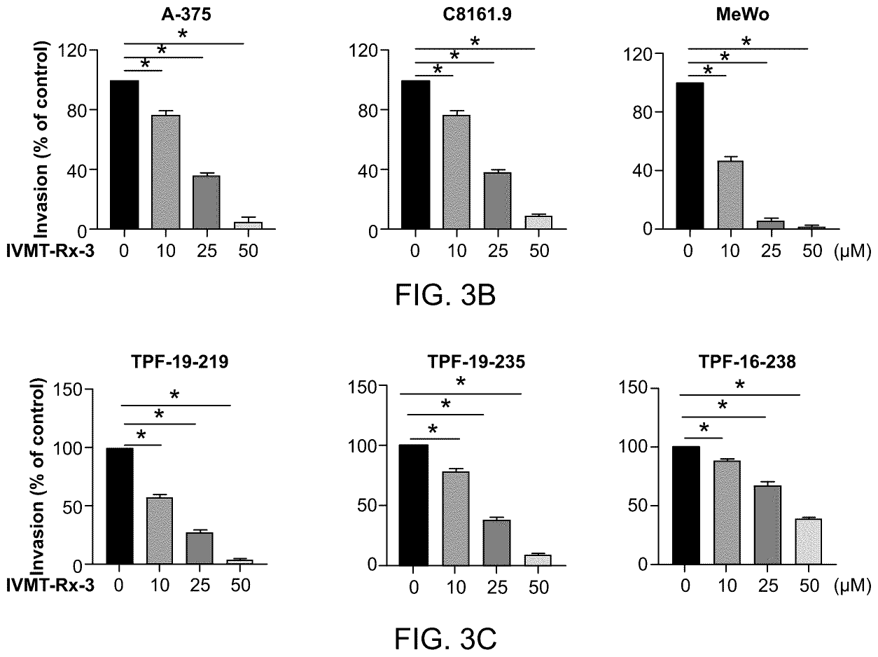

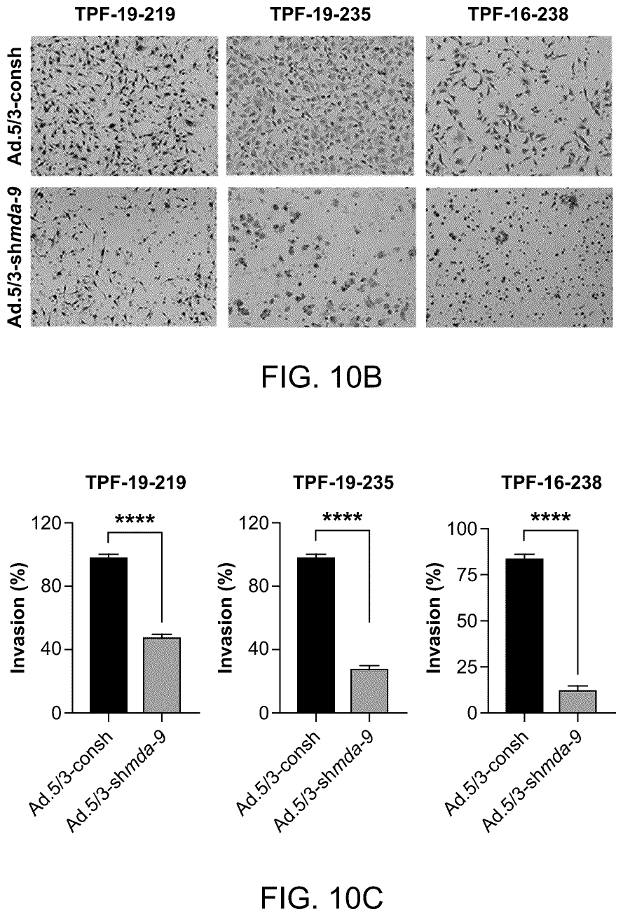

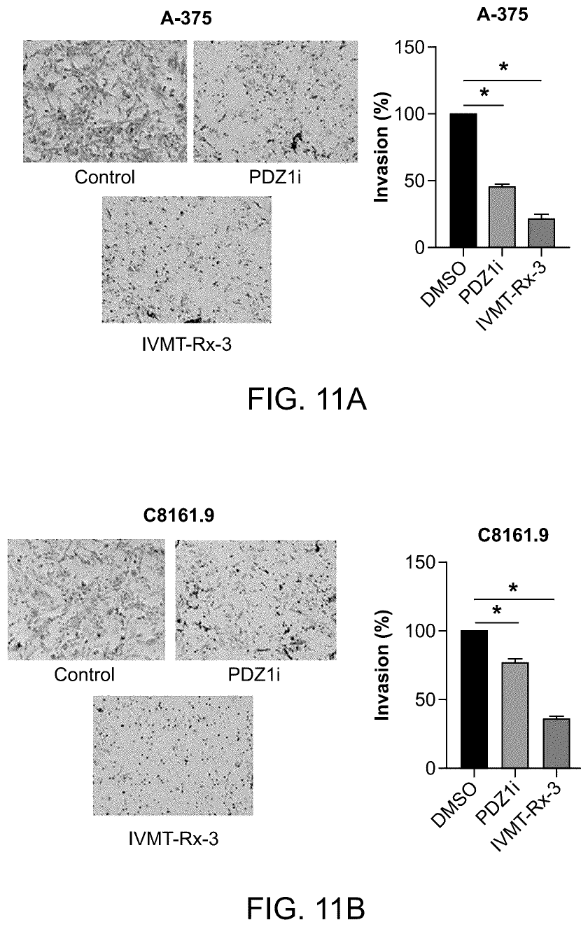

Given that invasion is one of the major determinants of metastasis progression and MDA-9/Syntenin was shown previously to positively regulate cellular invasion (6), the anti-invasive effects of IVMT-Rx-3 were tested. Pre-treatment with 25 μM, the intermediate dose of IVMT-Rx-3, significantly reduced invasion by 30 to 60% in different melanoma cell lines (FIGS. 3A-3C). The positive role of MDA-9/Syntenin in promoting invasion in these melanoma cell lines was confirmed through silencing of MDA-9/Syntenin using Ad.5/3-shmda-9 (FIGS. 10A-10C). Moreover, and as hypothesized, targeting both domains significantly enhanced the anti-invasive efficacy in comparison with PDZ1i (20) (FIG. 11A and FIG. 11B) without compromising non-toxic effects, justifying this as a potential chemical of choice for inhibiting MDA-9/Syntenin driven phenotypes. Since repeated treatment of IVMT-Rx-3 at higher concentrations (50 μM) did not show any effect on cell proliferation (FIG. 2A and FIG. 2B), the possibility of any potential growth inhibitory effects were ruled out as causative factors in inhibiting invasion capability (FIGS. 3A-3C). Since the melanoma cell lines used in this study harbor both wild type BRAF V600 (C8161.9, MeWo) (30) and mutant BRAF V600E (A-375) (30), TPF-19-219, TPF-19-235, TPF-16-238), the results confirm that the anti-invasive effects of IVMT-Rx-3 are not restricted to specific subtypes of melanoma with or without p53 mutations.

### Biological Example 3—Effect of IVMT-Rx-3 on MDA-9/Syntenin-Src Interaction and Effects on Downstream Signaling

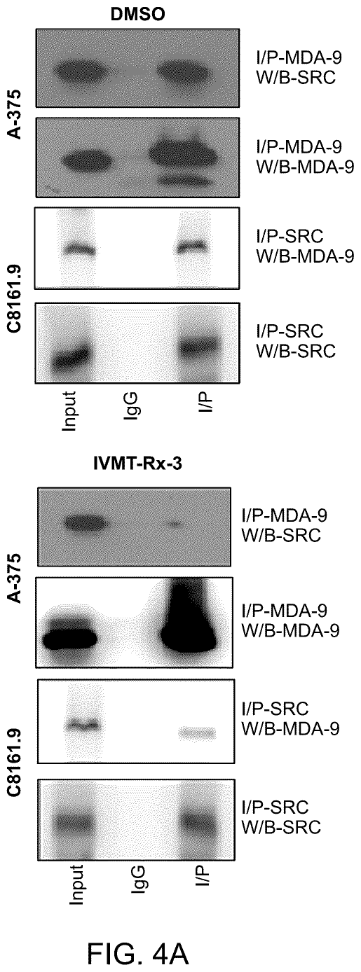

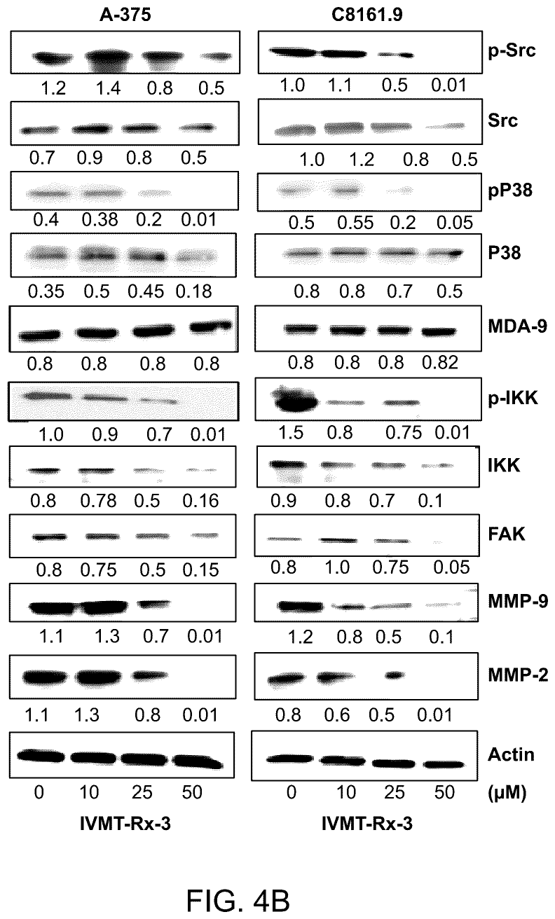

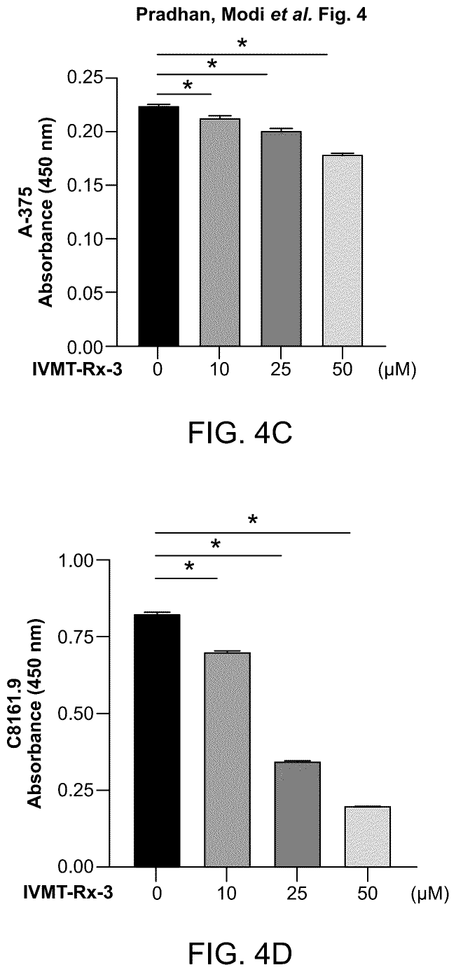

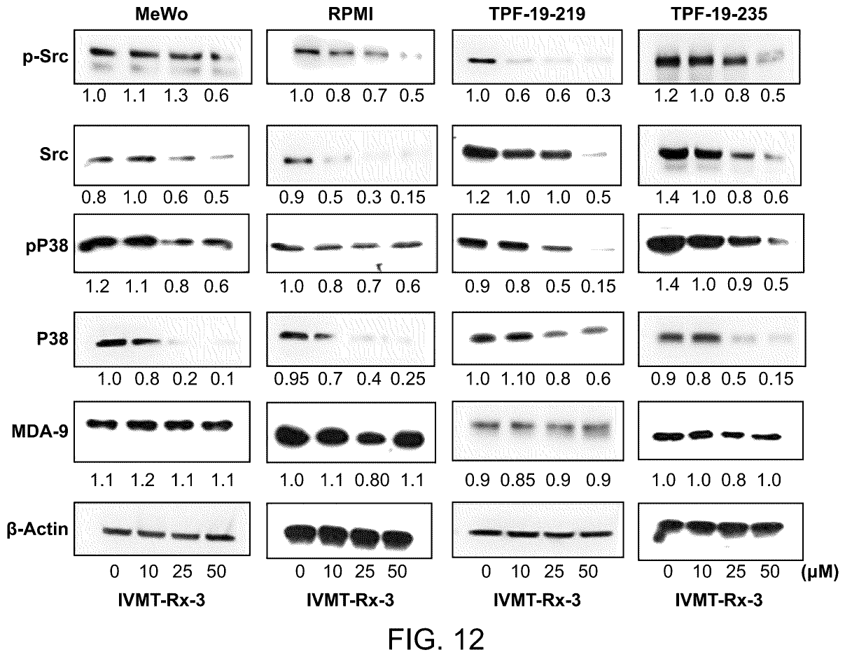

Given the established role of MDA-9/Syntenin and Src interaction in NF-κB activation (7), the effect of IVMT-Rx-3 on MDA-9/Syntenin and Src interaction was explored. Cells were treated and lysates were subjected to co-IP analysis. As predicted, treatment with IVMT-Rx-3 disrupted MDA-9/Syntenin: Src interactions (FIG. 4A). Since the physical interaction of MDA-9/Syntenin and Src is critical for Src auto-phosphorylation (7) and induction of downstream signaling, e.g., P38 MAPK phosphorylation followed by NF-κB activation (6,8,9), these signaling events were analyzed using Western blotting. Treatment with IVMT-Rx-3 significantly reduced Src activation, as evidenced by downregulation of Src phosphorylation in A-375 and C8161.9 cells (FIG. 4B). Reduced levels of p-P38, p-Src, and pIκB in IVMT-Rx-3-treated cells (FIG. 4B) were also found, all of which reflect inactivation of MDA-9/Syntenin downstream target pathway proteins. The inactivation signaling profiles in other melanoma cells was reconfirmed (FIG. 12), emphasizing the uniform bioactivity of IVMT-Rx-3 across multiple melanoma cell lines. MDA-9/Syntenin physically interacts with Src and amplifies NF-κB signaling in melanoma (6) that is critical for regulating the invasion phenotype. Accordingly, whether IVMT-Rx-3 treatment would inhibit NF-κB activity in melanoma cells was tested. IVMT-Rx-3 suppressed the transcriptional activity of NF-κB in a dose-dependent manner (FIGS. 4C and 4D).

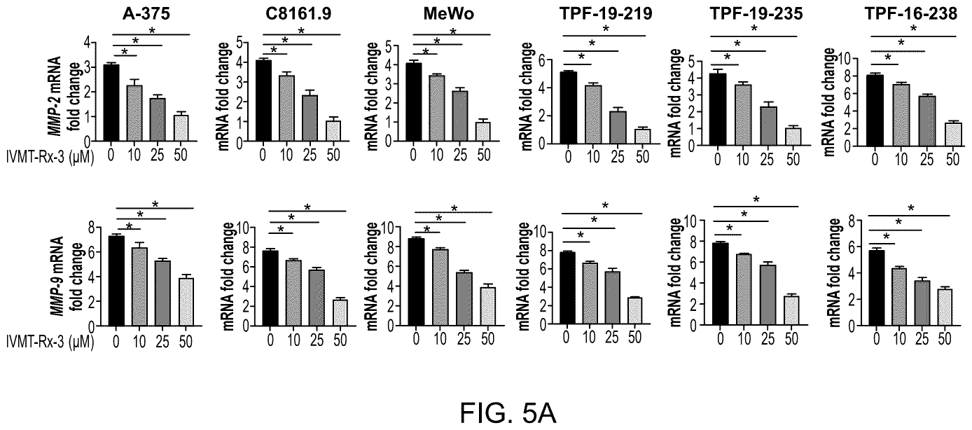

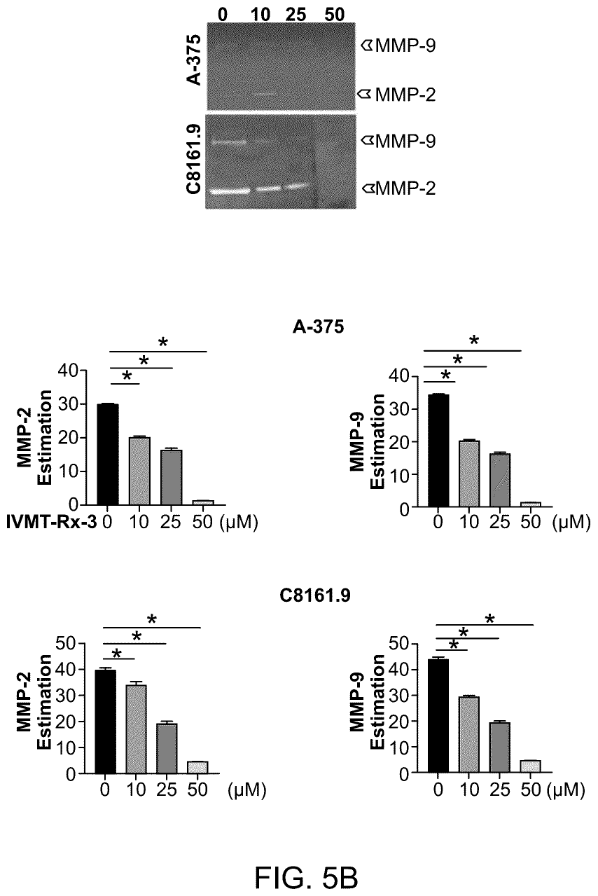

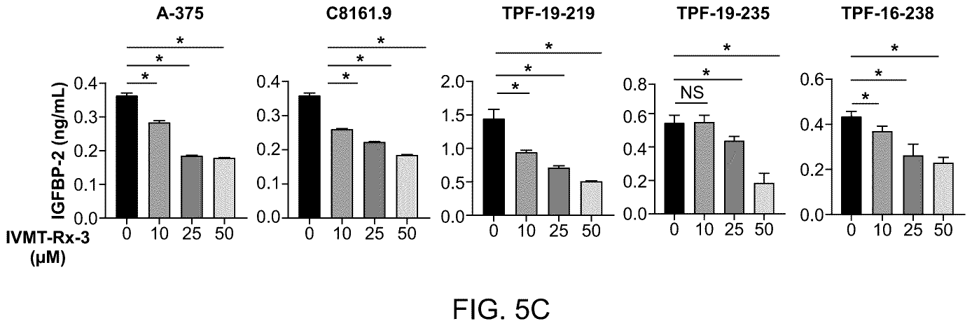

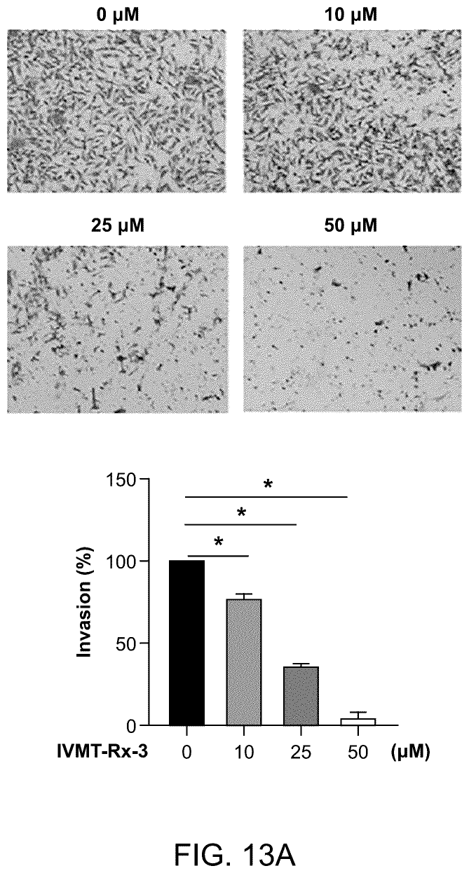

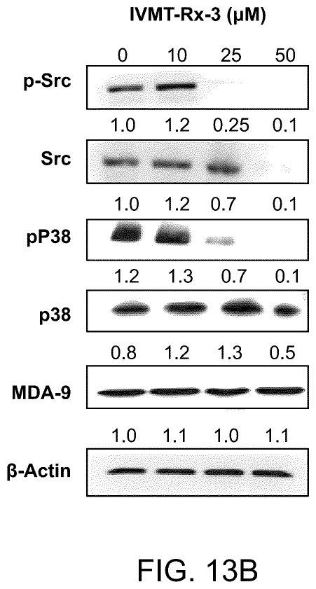

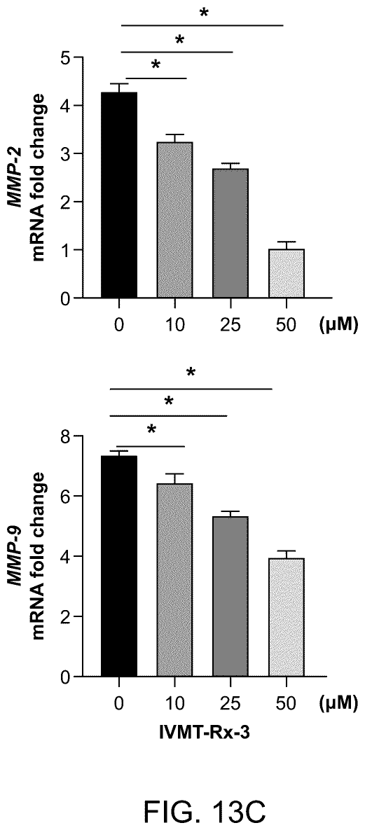

Matrix metalloproteinase 2 (MMP-2) or 9 (MMP-9), are two well-studied members of the MMP family that significantly impact cancer cell invasion (31). Multiple transcription factors could transcriptionally regulate the expression of MMP-2 and MMP-9 (31). Prior studies revealed (8,32) the importance of the MDA-9/Syntenin/NF-κB/MMPs axis in promoting cellular invasion (8). A dose-dependent IVMTRx-3-mediated down regulation of MMP-2 and MMP-9 expression, at both transcriptional (FIG. 5A) and translational levels (FIG. 4B), further confirmed the relevance of this axis pharmacologically. Also, to validate MMP activities, a zymography assay using conditioned media (FIG. 5B) was performed, documenting lower activity in treated cells. Similar findings including inhibition of invasion (FIG. 13A), downregulation of Src and downstream P38 (FIG. 13B), and lower expression of MMP-2 and MMP-9 were observed in the mouse melanoma cell line, i.e., B16 (FIG. 13C), rationalizing the use of this murine cell line in a syngeneic mouse model for in vivo validation of IVMT-Rx-3. A prior study documented a cell non-autonomous function of MDA-9/Syntenin by augmenting expression and secretion of pro-angiogenic and/or immune modulatory cytokines/growth factors (23), which may provide a complementary way to promote metastasis (9).

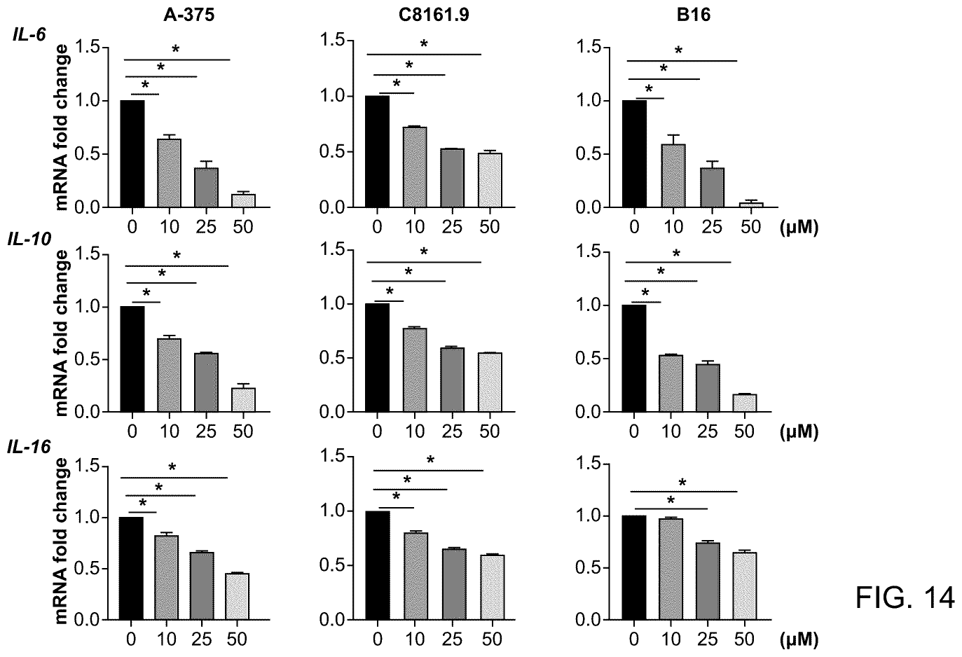

Insulin Growth Factor Binding Protein-2 (IGFBP-2) has been found in higher levels in the serum of individuals with diverse types of pathological conditions and is very germane in the context of angiogenesis, tumor promotion and chemoresistance (33). Since Src activation is critical for IGFBP-2 induced angiogenesis, a potential suppressive function of IVMT-Rx-3 on IGFBP-2 expression levels was investigated (9). Melanoma cells were treated with different doses (10, 25 or 50 μM) of IVMT-Rx-3 for 24 hr. and levels of IGFBP-2 in the media were determined using ELISA (FIG. 5C). An IVMT-Rx-3 dose-dependent downregulation of IGFBP-2 was evident in tumor-derived media confirming the dependency of IGFBP-2 expression on MDA-9/Syntenin (9). Blocking MDA-9/Syntenin expression inhibits the secretion of cytokines/growth factors in breast cancer resulting in the conversion of an immunosuppressive to immunostimulatory microenvironment (23). Accordingly, the level of specific melanoma metastasis-associated cytokines, e.g., IL-6, IL-10, IL-16, following treatment with IVMT-Rx-3 was analyzed. Down-regulation of these cytokines were observed by semiquantitative RT-PCR in both human and murine melanoma cells (FIG. 14). In total, it was demonstrated that IVMT-Rx-3 disrupts MDA-9/Syntenin: Src interaction-mediated signaling, which ultimately reduces activation of the transcription factor NF-κB and its downstream multiple effector proteins that non-autonomously regulate metastasis.

### Biological Example 4—Effect of IVMT-Rx-3 Alone and in Combination with α-PD-L1 Metastatic Melanoma

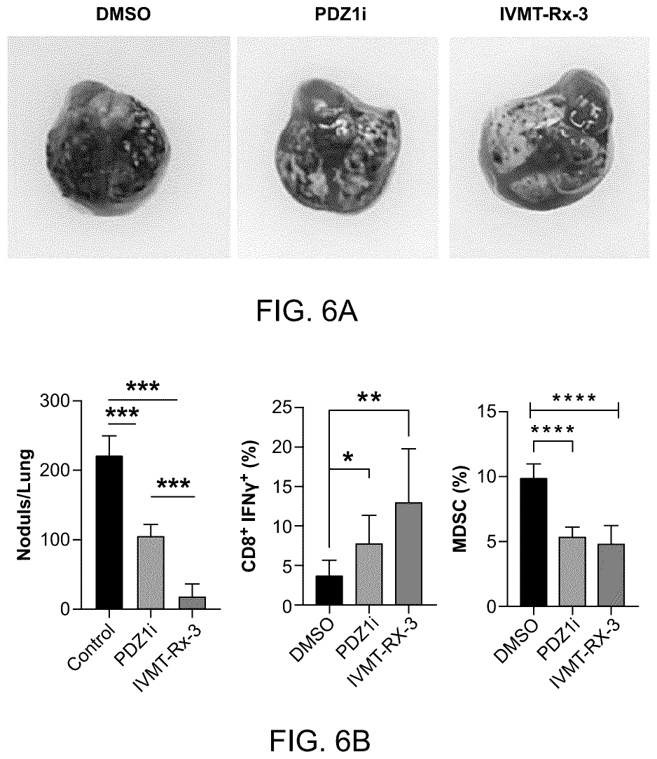

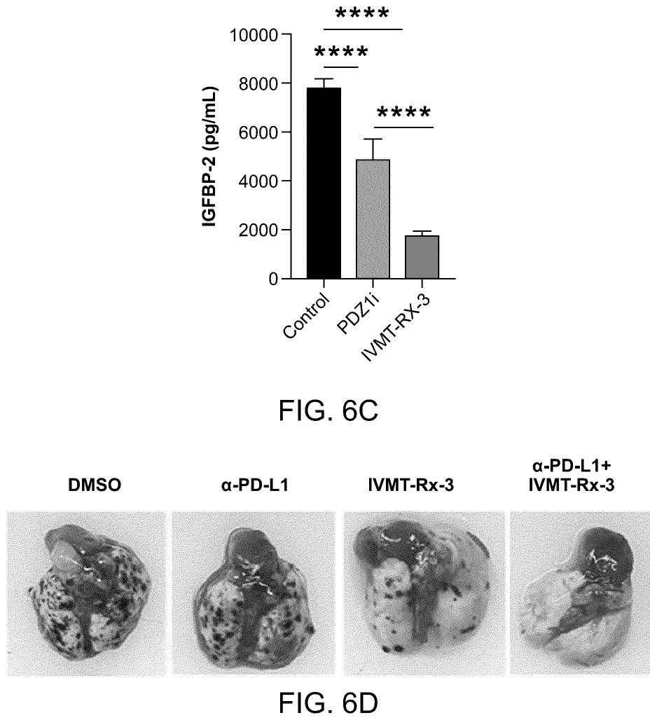

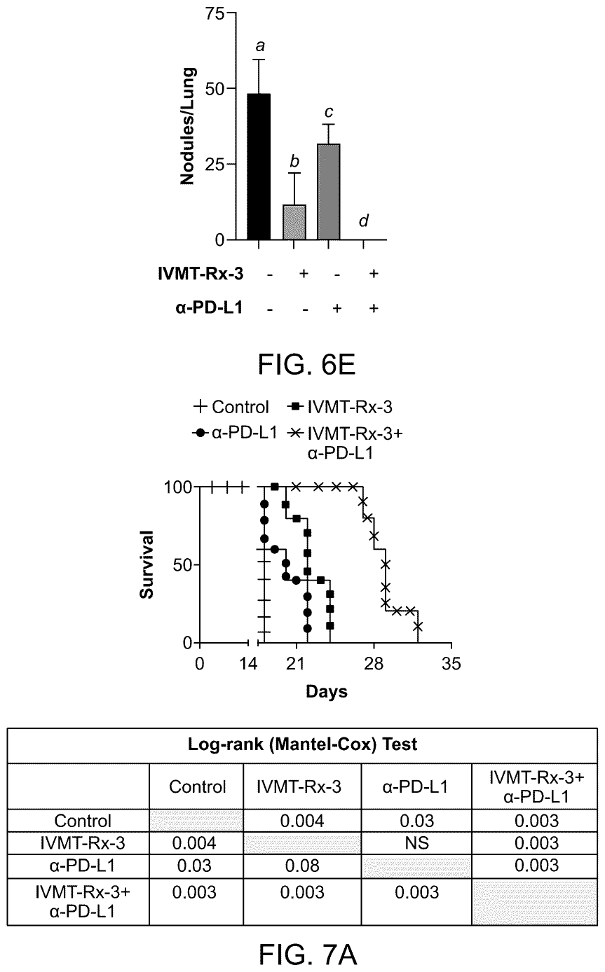

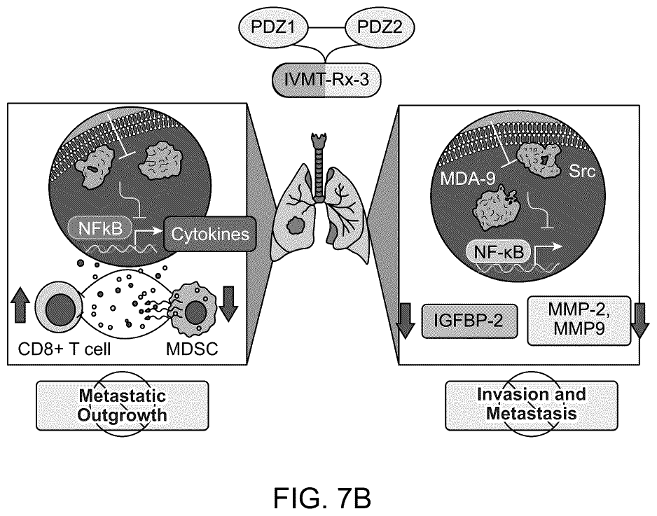

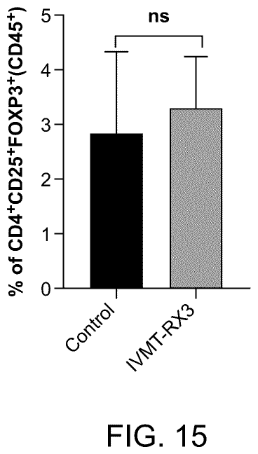

To determine if administering IVMT-Rx-3 could affect experimental metastasis in immunocompetent mice, B16 cells, a highly aggressive metastatic mouse melanoma, that results in lung nodules within 3 weeks following intravenous injection, were used. In this model, repeated treatment I.P. (3× per week, total of 6 injections) with the small molecule IVMT-Rx-3 significantly reduced the number of lung nodules (FIG. 6A) in comparison with control animals, which received vehicle. This experiment confirmed that treatment with IVMT-Rx-3 targeting MDA-9/Syntenin can directly prevent formation of lung metastases in vivo and the efficacy was significantly higher compared with PDZ1i. This effect correlated with a significant increase in IFN-γ-producing CD8⁺ T cell populations (FIG. 6B) and suppression of MDSCs supporting potential role of MDA-9/Syntenin in tumor immune modulation (FIG. 6B). There were no significant changes in the Treg population (FIG. 15). A down regulation of IGFBP-2 levels in blood plasma in IVMT-Rx-3 treated animals was observed (FIG. 6C), corroborating previous data (FIG. 5C). Liu et al. (34) showed that tumoral mda-9/syntenin expression correlated with anti-PD-L1 treatment effectiveness. There is a strong link between MDA-9/Syntenin and PD-L1 expression (34). Also, previous studies have shown that MDA-9/Syntenin expression is higher in tumor samples in comparison with normal tissue (6). In view of this, the combinatorial effect of targeting mda-9/syntenin with an α-PD-L1 checkpoint inhibitor was studied. The murine B16 melanoma model in C57BL6 animals with an intact immune system was used to investigate the effects of combinatorial therapy involving IVMT-Rx-3 and α-PD-L1 (FIG. 6D). IVMT-Rx-3 markedly reduced lung metastasis, and the combination treatment with PD-L1 further improved suppression of the metastatic phenotype (FIG. 6D), which enhanced the survival benefit of the combinatorial approach (FIG. 7A). The p values from t test as well as rank test showed significant difference. This therapeutic approach can be a potential strategy for improving the prognosis of patients with melanoma.

Therapeutic intervention in patients with advanced metastatic melanoma is a formidable clinical challenge requiring innovative strategies for identifying appropriate potentially “druggable” therapeutic targets to achieve positive patient outcomes. The lethality of melanoma metastasis and the ultimate failure of many/most therapies demand new strategies (6,7). Considering the elevated expression of MDA-9/Syntenin in multiple cancers including metastatic melanoma there is support for this gene/protein being a viable molecular target, which has been validated genetically and pharmacologically (35). This disclosure herein focuses on IVMT-Rx-3, a new MDA-9/Syntenin-antagonist, that inhibits tumor cell invasion more effectively than PDZ1i, facilitates immune cytotoxicity, and synergizes with immunotherapy (Summarized in FIG. 7B).

Multiple or tandem PDZ domains are important in canonically defining proper protein folding, yet in many instances this relationship is not well-defined and unfortunately no general mechanisms are established that apply universally to predict this process (36). Deletion of a single PDZ domain has been shown to be sufficient to inhibit cancer progression mediated by MDA-9/Syntenin. (20-23). Although this hypothesis was validated using genetic (shmda-9 constructs) and pharmacological (PDZ1i) approaches, this over simplified view does not explain potential cooperative effects of the two tandem PDZ domains of MDA-9/Syntenin or other similar proteins following interaction with their partners. Using four peptides in a cell free system Grembecka et al. (34) demonstrated that all four peptides bound to both domains with variable affinities (36), signifying the complexity of PDZ domain structures. In contrast, multiple proteins have selective preferences for specific PDZ domains, e.g., IGF-1R (32) or EGFR (11) for PDZ1, and Src for PDZ2 (7), as demonstrated using coimmunoprecipitation assays adding an additional level of complexity to PDZ domain targeting for creating protein networks thereby making these domains far less attractive for drug development. This supposition is now changing based on recent success in uniquely targeting either or both domains (17,37).

Initial attempts to target PDZ domains have generally been peptide-based, i.e., a short amino acids consisting of either modified peptides, including thioketone, or unnatural sequences from the C-terminal residues of endogenous partners (38). More recently, a series of small molecule inhibitors, identified utilizing various screening approaches that include in silico design, NMR and FBDD guided with NMR, have also been discovered and successfully assayed for biological activities (reviewed by Hoffer at al. (39)). Liu et al. developed dimeric peptides using natural ligands with an appropriately spaced linker and demonstrated binding simultaneously with the tandem PDZ domains thereby blocking cell migration/proliferation/spreading (24). The PDZ1 domain was targeted and the specificity and bioactivities tested in terms of anti-invasion and anti-metastatic activities in multiple cancers (20,22). Very recently, using in silico design PDZ2 domain targeting small molecules were generated and evaluated in exosome biogenesis (25). Considering all of these documented approaches, the disclosure herein is unique in that it uses both a rationally-developed small molecule PDZ1 inhibitor (PDZ1i) and a natural ligand for the PDZ2 domain creating a bivalent molecule that targets both PDZ domains thereby providing broader and more global PDZ coverage and inhibition.

Although multiple PDZ domains have been targeted by small molecules and peptides, weak affinities for their targets remain a potential problem. Recent studies by Deng et al. (40) suggest that multivalent interactions have the ability to increase the binding affinity and specificity of ligands to their targets. They showed that ligands containing two specific binding domains could enhance binding affinity and specificity, reduce the effective concentration range, and have a robust concentration dependency. Bivalency can also enhance targeting, reducing the risk of non-specificity and off-target toxicity. IVMT-Rx-3 does not have enhanced affinity as compared with PDZ1i (K_{d}=23 μM) or other peptide-based inhibitors (K_{d}=0.21 μM) (24), although IVMT-Rx-3 demonstrates significant enhancement in suppression of the transformed phenotype as compared with PDZ1i. This again illustrates the complexity of interacting molecules and the fact that the binding affinities are not always predictive of therapeutic efficacy (42). Thus, therapeutic efficacy of IVMT-Rx-3 is not directly associated with its moderate binding affinity. Further studies are ongoing to decipher the overall impact of IVMT-Rx-3 on suppressing multiple stages of cancer progression and metastasis.

The mechanisms by which MDA-9/Syntenin orchestrates cellular invasion are being resolved (17). One notable example of a transcription factor directly regulating cellular invasion is NFκB, where stimulation is obligatory to activate the expression of MMP-2 and MMP-9 that mediates invasiveness (6). As predicated, IVMT-Rx-3 driven NFκB inactivation decreased the expression and activities of MMP-2/MMP-9 and impaired this key metastatic event. The NFκB pathway also stimulates expression of tumor cell intrinsic pro-inflammatory mediators that enhance influx of various immune suppressive mediators that modify the immune landscape (43). Treatment of melanoma cells with IVMT-Rx-3 resulted in downregulation of various chemokines, some of which play a direct role in melanoma progression. For example, higher expression of IL-10 is correlated with metastatic melanoma progression (44). Moreover, IL-6 is expressed at higher levels in metastatic melanoma patients (45), augments immune suppression potentially through regulation of MDSC differentiation in a murine model (46) and is a prognostic factor for survival (46). In these contexts, targeting this signaling cascade would be beneficial in eliciting immune suppression, also examined in the current study.

A mechanism by which tumor cells bypass immune recognition involves immune editing, which restrains the cytotoxic effect of immune cells (47). PD-L1 expression in tumor cells and interaction with PD1 are essential for immune suppression and are associated with objective responses to immunotherapy (48,49) with recent data indicating that targeted therapies may enhance PD-L1 expression. Targeting MDA-9/Syntenin using PDZ1i (20-23) and IVMT-Rx-3 causes T cell infiltration into the tumor environment, which can positively select immune-resistant tumor cells through IFNγ-induced PD-L1 expression. Thus, one could hypothesize that a combined targeted therapeutic checkpoint inhibitor might act synergistically and prolong therapeutic responses, a model supported by our study. These results demonstrate a novel way of combining a small molecule and immunotherapy, which has been used recently to treat metastatic melanoma (anti-PD1 inhibitors plus BRAF+/−MEK inhibitors), resulting in significant activity and manageable toxicity (46).

An objective of clinical medicine is to develop personalized strategies that combine effective treatment and monitoring an individual patient's response to therapy. Determining expression levels of MDA-9/Syntenin in biopsy samples from patients provides an analytical method to stratify patients who likely to benefit from IVMT-Rx-3 therapy. However, using this approach for monitoring therapeutic responses highlights a potential challenge since IVMT-Rx-3 does not affect MDA-9/Syntenin protein expression making this approach impractical. To overcome this obstacle, an MDA-9/Syntenin downstream effector protein, IGFBP-2 (9,32), is being used, which can be monitored non-invasively using a laboratory-based ELISA approach. The diagnostic value for IGFBP-2 was previously reported (33) and its further application as a pharmacodynamic biomarker has been extended for targeted therapeutics. A previously reported drug-diagnostic co-development model (50) would provide a “companion diagnostic” which needs additional analytical validation, e.g., stratifying with disease stage or generating multi-biomarker panels for enhancing specificity and sensitivity.

Stromal MDA-9/Syntenin plays a pivotal role in facilitating melanoma metastasis (18). Although MDA-9/Syntenin expression in melanoma cells is important in inducing invasion and migration (17), optimal metastasis also requires expression of this protein in the stromal compartment. It is speculated that expression of mda-9/Syntenin in stroma may afford melanoma cells a hospitable niche and targeting both the tumor and stroma with a small molecule would effectively block the contributions of both compartments to metastasis eliciting a robust anti-metastatic outcome. In total, the dual-targeting approach disclosed herein provides further support for the hypothesis that MDA-9/Syntenin expression has broad spectrum contributory roles in metastasis, from both a tumor and microenvironmental perspective. Further comprehensive investigation of IVMT-Rx-3 including in vivo PK and safety assessment in order to understand the physiochemical properties and risk profile, PK/PD modeling to correlate the concentration of active chemicals at the primary tumor and metastatic sites, developing a clinically relevant formulation for intravenous or oral administration would be important steps in potentially progressing IVMT-Rx-3 from bench to bedside. The examples disclosed herein also show that IVMT-Rx-3 can be used in addition to conventional immunotherapeutic agents to enhance efficacy against melanoma.

### REFERENCES

1. Schadendorf D, Berking C, Griewank K G, Gutzmer R, Hauschild A. Melanoma. The Lancet 2018; 392 (10151): 971-84.
2. Huang F W, Hodis E, Xu M J, Kryukov G V, Chin L, Garraway L A. Highly recurrent TERT promoter mutations in human melanoma. Science 2013; 339 (6122): 957-9.
3. Hodis E, Watson I R, Kryukov G V, Arold S T, Imielinski M, Theurillat J P, et al. A landscape of driver mutations in melanoma. Cell 2012; 150 (2): 251-63.
4. Lin J J, Jiang H, Fisher P B. Melanoma differentiation associated gene-9, mda-9, is a human gamma interferon responsive gene. Gene 1998; 207 (2): 105-10.
5. Lin J J, Jiang H P, Fisher P B. Characterization of a novel melanoma differentiation-associated gene, mda-9, that is down-regulated during terminal cell differentiation. Molecular and Cellular Differentiation 1996; 4 (4): 317-33.
6. Boukerche H, Su Z Z, Emdad L, Baril P, Balme B, Thomas L, et al. mda-9/Syntenin: a positive regulator of melanoma metastasis. Cancer Res 2005; 65 (23): 10901-11.
7. Boukerche H, Su Z Z, Prevot C, Sarkar D, Fisher P B. mda-9/Syntenin promotes metastasis in human melanoma cells by activating c-Src. Proc Natl Acad Sci USA 2008; 105 (41): 15914-9.
8. Das S K, Bhutia S K, Sokhi U K, Azab B, Su Z Z, Boukerche H, et al. Raf kinase inhibitor RKIP inhibits MDA-9/syntenin-mediated metastasis in melanoma. Cancer Res 2012; 72 (23): 6217-26.
9. Das S K, Bhutia S K, Azab B, Kegelman T P, Peachy L, Santhekadur P K, et al. MDA-9/Syntenin and IGFBP-2 Promote Angiogenesis in Human Melanoma. Cancer Res 2013; 73 (2): 844-54.
10. Bacolod M D D S, Sokhi U K, Bradley S, Fenstermacher D A, Pellecchia M, Emdad L, Sarkar D, Fisher P B. Examination of Epigenetic and other Molecular Factors Associated with mda-9/Syntenin Dysregulation in Cancer Through Integrated Analyses of Public Genomic Datasets. Adv Cancer Res 2015; 127:49-121.
11. Dasgupta S, Menezes M E, Das S K, Emdad L, Janjic A, Bhatia S, et al. Novel role of MDA-9/syntenin in regulating urothelial cell proliferation by modulating EGFR signaling. Clin Cancer Res 2013; 19 (17): 4621-33.
12. Kim W Y, Jang J Y, Jeon Y K, Chung D H, Kim Y G, Kim C W. Syntenin increases the invasiveness of small cell lung cancer cells by activating p38, AKT, focal adhesion kinase and SP1. Exp Mol Med 2014; 46.
13. Liu X, Zhang X, Lv Y, Xiang J, Shi J. Overexpression of syntenin enhances hepatoma cell proliferation and invasion: potential roles in human hepatoma. Oncol Rep 2014; 32 (6): 2810-6.
14. Hwangbo C, Park J, Lee J H. mda-9/Syntenin protein positively regulates the activation of Akt protein by facilitating integrin-linked kinase adaptor function during adhesion to type I collagen. J Biol Chem 2011; 286 (38): 33601-12.
15. Hwangbo C, Tae N, Lee S, Kim O, Park O K, Kim J. et al. Syntenin regulates TGF-betal-induced Smad activation and the epithelial-to-mesenchymal transition by inhibiting caveolin-mediated TGF-beta type I receptor internalization. Oncogene 2016; 35 (3): 389-401.
16. Jeon H Y, Das S K, Dasgupta S, Emdad L, Sarkar D, Kim S H, et al. Expression patterns of MDA-9/syntenin during development of the mouse embryo. J Mol Histol 2013; 44 (2): 159-66.
17. Das S K, Maji S, Wechman S L, Bhoopathi P, Pradhan A K, Talukdar S, et al. MDA-9/Syntenin (SDCBP): Novel gene and therapeutic target for cancer metastasis. Pharmacol Res 2020; 155:104695.
18. Das S K, Guo C, Pradhan A K, Bhoopathi P, Talukdar S, Shen X N, et al. Knockout of MDA-9/Syntenin (SDCBP) expression in the microenvironment dampens tumor-supporting inflammation and inhibits melanoma metastasis. Oncotarget 2016; 7 (30): 13.
19. Tamura K, Ikutani M, Yoshida T, Tanaka-Hayashi A, Yanagibashi T, Inoue R, et al. Increased production of intestinal immunoglobulins in Syntenin-1-deficient mice. Immunobiology 2015; 220 (5): 597-604.
20. Kegelman T P, Wu B, Das S K, Talukdar S, Beckta J M, Hu B, et al. Inhibition of radiation-induced glioblastoma invasion by genetic and pharmacological targeting of MDA-9/Syntenin. Proc Natl Acad Sci USA 2017; 114 (2): 370-5.
21. Das S K, Kegelman T P, Pradhan A K, Shen X N, Bhoopathi P, Talukdar S, et al.

Suppression of prostate cancer pathogenesis using an MDA-9/Syntenin (SDCBP) PDZ1 small molecule inhibitor. Mol Cancer Ther 2019; 18: (11): 1997-2007.
22. Bhoopathi P, Pradhan A K, Bacolod M D, Emdad L, Sarkar D, Das S K, et al. Regulation of neuroblastoma migration, invasion, and in vivo metastasis by genetic and pharmacological manipulation of MDA-9/Syntenin. Oncogene 2019; 38:6781-6793.
23. Pradhan A K, Maji S, Bhoopathi P, Talukdar S, Mannangatti P, Guo C, et al.

Pharmacological inhibition of MDA-9/Syntenin blocks breast cancer metastasis through suppression of IL-1beta. Proc Natl Acad Sci USA 2021; 118 (21).
24. Liu J, Qu J, Zhou W, Huang Y, Jia L, Huang X, et al. Syntenin-targeted peptide blocker inhibits progression of cancer cells. Eur J Med Chem 2018; 154:354-66.
25. Leblanc R, Kashyap R, Barral K, Egea-Jimenez A L, Kovalskyy D, Feracci M, et al. Pharmacological inhibition of syntenin PDZ2 domain impairs breast cancer cell activities and exosome loading with syndecan and EpCAM cargo. J Extracell Vesicles 2020; 10 (2): e12039.
26. Garcia M, Hoffer L, Leblanc R, Benmansour F, Feracci M, Derviaux C, et al. Fragment-based drug design targeting syntenin PDZ2 domain involved in exosomal release and tumour spread. Eur J Med Chem 2021; 223:113601.
27. Ralli M, Botticelli A, Visconti I C, Angeletti D, Fiore M, Marchetti P, et al. Immunotherapy in the Treatment of Metastatic Melanoma: Current Knowledge and Future Directions. J Immunol Res 2020; 2020:9235638.
28. Vanneman M, Dranoff G. Combining immunotherapy and targeted therapies in cancer treatment. Nat Rev Cancer 2012; 12 (4): 237-51.
29. Emdad L, Lee S G, Su Z Z, Jeon H Y, Boukerche H, Sarkar D, et al. Astrocyte elevated gene-1 (AEG-1) functions as an oncogene and regulates angiogenesis. Proc Natl Acad Sci USA 2009; 106 (50): 21300-5.
30. Xu M J, Cooke M, Steinmetz D, Karakousis G, Saxena D, Bartlett E, et al. A novel approach for the detection and genetic analysis of live melanoma circulating tumor cells. PLOS One 2015; 10 (3): e0123376.
31. Quintero-Fabian S, Arreola R, Becerril-Villanueva E, Torres-Romero J C, Arana-Argaez V, Lara-Riegos J, et al. Role of Matrix Metalloproteinases in Angiogenesis and Cancer. Front Oncol 2019; 9:1370.
32. Das S K, Pradhan A K, Bhoopathi P, Talukdar S, Shen X N, Sarkar D, et al. The MDA-9/Syntenin/IGF1R/STAT3 Axis Directs Prostate Cancer Invasion. Cancer Res 2018; 78 (11): 2852-63.
33. Han S, Li Z, Master L M, Master Z W, Wu A. Exogenous IGFBP-2 promotes proliferation, invasion, and chemoresistance to temozolomide in glioma cells via the integrin betal-ERK pathway. Br J Cancer 2014; 111 (7): 1400-9.
34. Liu J, Yang Y, Wang H, Wang B, Zhao K, Jiang W, et al. Syntenin1/MDA-9 (SDCBP) induces immune evasion in triple-negative breast cancer by upregulating PD-L1. Breast Cancer Res Treat 2018; 171 (2): 345-57 doi 10.1007/s10549-018-4833-8.
35. Das S K, Sarkar D, Emdad L, Fisher P B. MDA-9/Syntenin: An emerging global molecular target regulating cancer invasion and metastasis. Adv Cancer Res 2019; 144:137-91.
36. Grembecka J, Cierpicki T, Devedjiev Y, Derewenda U, Kang B S, Bushweller J H, et al. The binding of the PDZ tandem of syntenin to target proteins. Biochemistry 2006; 45 (11): 3674-83.
37. Pradhan A K M S, Das S K, Emdad L, Sarkar D, Fisher P B. MDA-9/Syntenin/SDCBP: new insights into a unique multifunctional scaffold protein. Cancer Metastasis Rev 2020; 39 (3): 769-81.
38. Bach A, Chi C N, Olsen T B, Pedersen S W, Roder M U, Pang G F, et al. Modified peptides as potent inhibitors of the postsynaptic density-95/N-methyl-D-aspartate receptor interaction. J Med Chem 2008; 51 (20): 6450-9.
39. Hoffer L, Roche P, Morelli X. Rational Design of PDZ Domain Inhibitors: Discovery of Small Organic Compounds Targeting P D Z Domains. Methods Mol Biol 2021; 2256:277-89.
40. Deng Y, Efremov A K, Yan J. Modulating binding affinity, specificity, and configurations by multivalent interactions. Biophys J 2022; 121 (10): 1868-80.
41. Christensen N R, De Luca M, Lever M B, Richner M, Hansen A B, Noes-Holt G, et al. A highaffinity, bivalent PDZ domain inhibitor complexes PICKI to alleviate neuropathic pain. EMBO Mol Med 2020; 12 (6): e11248.
42. Mestres J, Gregori-Puigjane E. Conciliating binding efficiency and polypharmacology. Trends Pharmacol Sci 2009; 30 (9): 470-4.
43. Hui Yu L L, Zhiqiang Zhang, Huiyuan Zhang & Hongbo Hu Targeting N F-κB pathway for the therapy of diseases: mechanism and clinical study. Sig Transduct Target Ther 2020; 5 (209).
44. Itakura E, Huang R R, Wen D R, Paul E, Wunsch P H, Cochran A J. IL-10 expression by primary tumor cells correlates with melanoma progression from radial to vertical growth phase and development of metastatic competence. Mod Pathol 2011; 24 (6): 801-9.
45. Laino A S, Woods D, Vassallo M, Qian X, Tang H, Wind-Rotolo M, et al. Serum interleukin-6 and C-reactive protein are associated with survival in melanoma patients receiving immune checkpoint inhibition. J Immunother Cancer 2020; 8 (1).
46. Samlowski W, Adajar C. Cautious addition of targeted therapy to PD-1 inhibitors after initial progression of BRAF mutant metastatic melanoma on checkpoint inhibitor therapy. BMC Cancer 2021; 21 (1): 1187.
47. Nurieva R, Wang J, Sahoo A. T-cell tolerance in cancer. Immunotherapy 2013; 5 (5): 513-31.
48. Tsai K K, Zarzoso I, Daud A I. PD-1 and PD-L1 antibodies for melanoma. Human vaccines & immunotherapeutics 2014; 10 (11): 3111-6.
49. Simeone E, Ascierto P A. Anti-PD-1 and PD-L1 antibodies in metastatic melanoma. Melanoma management 2017; 4 (4): 175-8.
50. Jorgensen J T, Hersom M. Companion diagnostics—a tool to improve pharmacotherapy. Ann Transl Med 2016; 4 (24): 482.

### INCORPORATION BY REFERENCE

All publications and patents cited throughout the specification herein (including all patents, patent applications, scientific publications, manufacturers' specifications, instructions, etc.), whether supra or infra, are hereby incorporated by reference in their entirety for all purposes. To the extent the material incorporated by reference contradicts or is inconsistent with this specification, the specification will supersede any such material.

1. A compound of formula (I), 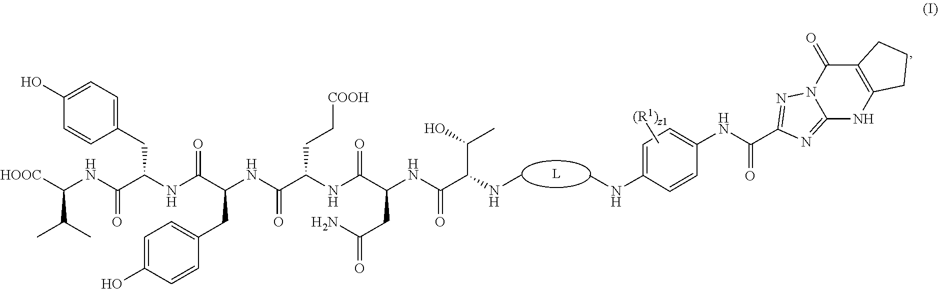
or a pharmaceutically acceptable salt thereof, wherein
L is a non-cleavable linker;
each R¹ is independently selected from halo, —OH, and C₁₋₆ alkyl; and
z1 is an integer from 0 to 3.

2. The compound of claim 1, wherein each R¹ is independently halo or C₁₋₆ alkyl.

3. The compound of claim 1 or 2, wherein z1 is 2.

4. The compound of claim 1, wherein the compound is a compound of formula (Ia), 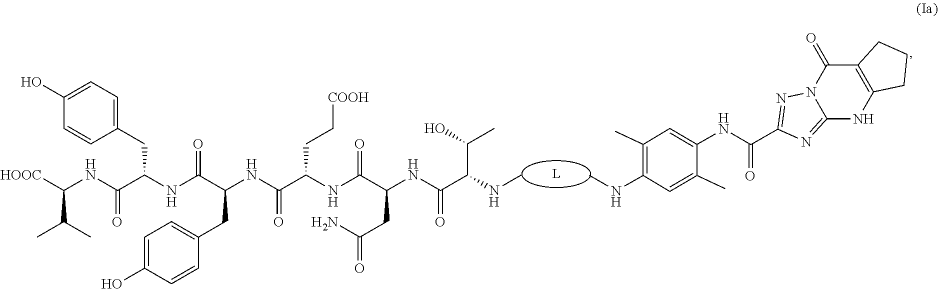 [SMILES: CN[C@H](C(=O)N[C@@H](CC(N)=O)C(=O)N[C@@H](CCC(=O)O)C(=O)N[C@@H](Cc1ccc(O)cc1)C(=O)N[C@@H](Cc1ccc(O)cc1)C(=O)N[C@H](C(=O)O)C(C)C)[C@@H](C)O ; CNc1cc(C)c(NC(=O)c2nc3[nH]c4c(c(=O)n3n2)CCC4)cc1C]
or a pharmaceutically acceptable salt thereof.

5. The compound of any one of claims 1-4, wherein L is at least 15 Angstroms in length.

6. The compound of any one of claims 1-4, wherein L is 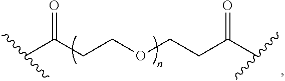
and wherein n is an integer from 5 to 10.

7. The compound of claim 6, wherein n is an integer from 5 to 7.

8. A compound represented by the formula 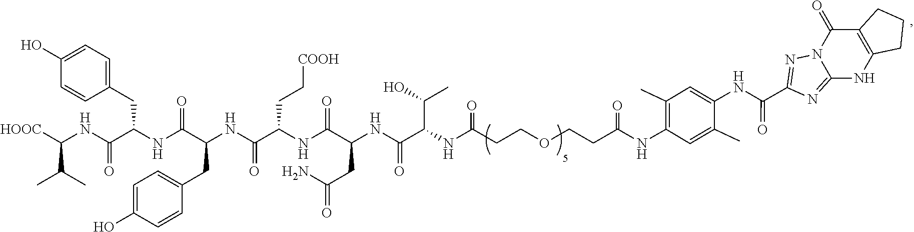
or a pharmaceutically acceptable salt thereof.

9. A compound represented by the formula

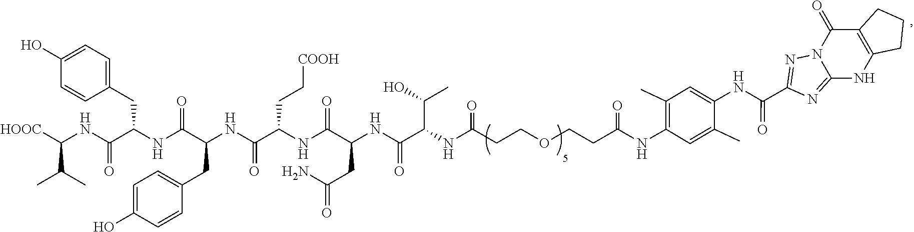

10. A pharmaceutical composition comprising the compound of any one of claims 1-9, or a pharmaceutically acceptable salt thereof, and one or more pharmaceutically acceptable excipients.

11. A method of treating cancer in a subject in need thereof, the method comprising administering to the subject an effective amount of the compound of any one of claims 1-9, or a pharmaceutically acceptable salt thereof, or the pharmaceutical composition of claim 10.

12. A method of treating cancer in a subject in need thereof, the method comprising administering to the subject an effective amount of the compound of any one of claims 1-9, or a pharmaceutically acceptable salt thereof or the pharmaceutical composition of claim 10, and an effective amount of an α-PD-L1 checkpoint inhibitor.

13. The method of claim 11 or 12, wherein the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma.

14. The method of claim 11 or 12, wherein the cancer is melanoma.

15. The method of claim 14, wherein the melanoma is metastatic melanoma.

16. The method of any one of claims 11-15, wherein treating the cancer comprises preventing or slowing the development of metastasis of the cancer.

17. The method of claim 14 or 15, wherein treating melanoma comprises preventing or slowing the development of melanoma lung metastases.

18. The method of any one of claims 14, 15, and 17, wherein the melanoma comprises a p53 mutation.

19. The method of any one of claims 14, 15, and 17, wherein the melanoma does not comprise a p53 mutation.

20. The method of any one of claims 11-19, wherein the treating comprises reducing nuclear factor kappa-light-chain-enhancer of activated B cells (NF-κB) signaling in the cancer by administering to the subject the compound of any one of claims 1-9 or the pharmaceutical composition of claim 10.

21. The method of any one of claims 11-20, wherein the treating comprises reducing matrix metalloproteinase 2 (MMP-2) and/or matrix metalloproteinase 9 (MMP-9) expression in the cancer by administering to the subject the compound of any one of claims 1-9 or the pharmaceutical composition of claim 10.

22. The method of any one of claims 11-21, wherein the treating comprises reducing insulin growth factor binding protein-2 (IGFBP-2) expression in the cancer by administering to the subject the compound of any one of claims 1-9 or the pharmaceutical composition of claim 10.

23. The method of any one of claims 11-22, wherein the treating comprises reducing interleukin-6, interleukin-10, and/or interleukin-16 expression in the cancer by administering to the subject the compound of any one of claims 1-9 or the pharmaceutical composition of claim 10.

24. The method of any one of claims 11-23, wherein the treating comprises increasing IFN-γ-producing CD8⁺ T cell populations in the subject by administering to the subject the compound of any one of claims 1-9 or the pharmaceutical composition of claim 10.

25. The method of any one of claims 11-24, wherein the treating comprises decreasing myeloid-derived suppressor cell (MDSC) populations in the subject by administering to the subject the compound of any one of claims 1-9 or the pharmaceutical composition of claim 10.

26. A method of preventing or slowing the development of cancer metastasis in a subject with cancer, the method comprising administering to the subject an effective amount of the compound of any one of claims 1-9 or the pharmaceutical composition of claim 10.

27. A method of preventing or slowing the development of cancer metastasis in a subject with cancer, the method comprising administering to the subject an effective amount of the compound of any one of claims 1-9 or the pharmaceutical composition of claim 10, and an effective amount of an α-PD-L1 checkpoint inhibitor.

28. The method of claim 26 or 27, wherein the cancer is selected from the group consisting of melanoma, glioblastoma, head and neck cancer, urothelial cancer, breast cancer, uveal melanoma, gastric cancer, lung adenocarcinoma, hepatocellular carcinoma, colorectal cancer, prostate cancer, pancreatic cancer, and neuroblastoma.

29. The method of claim 26 or 27, wherein the cancer is melanoma.

30. The method of claim 29, wherein the melanoma is metastatic melanoma.

31. The method of claim 29 or 30, wherein preventing or slowing the development of melanoma metastasis comprises preventing or slowing the development of melanoma lung metastases.

32. The method of any one of claims 29-31, wherein the melanoma comprises a p53 mutation.

33. The method of any one of claims 29-31, wherein the melanoma does not comprise a p53 mutation.

34. The method of any one of claims 26-33, wherein the preventing or slowing the development of the cancer comprises reducing nuclear factor kappa-light-chain-enhancer of activated B cells (NF-κB) signaling in the cancer by administering to the subject the compound of any one of claims 1-9 or the pharmaceutical composition of claim 10.

35. The method of any one of claims 26-34, wherein the preventing or slowing the development of the cancer comprises reducing matrix metalloproteinase 2 (MMP-2) and/or matrix metalloproteinase 9 (MMP-9) expression in the cancer by administering to the subject the compound of any one of claims 1-9 or the pharmaceutical composition of claim 10.

36. The method of any one of claims 26-35, wherein the preventing or slowing the development of the cancer comprises reducing insulin growth factor binding protein-2 (IGFBP-2) expression in the cancer by administering to the subject the compound of any one of claims 1-9 or the pharmaceutical composition of claim 10.

37. The method of any one of claims 26-36, wherein the preventing or slowing the development of the cancer comprises reducing interleukin-6, interleukin-10, and/or interleukin-16 expression in the cancer by administering to the subject the compound of any one of claims 1-9 or the pharmaceutical composition of claim 10.

38. The method of any one of claims 26-37, wherein the preventing or slowing the development of the cancer comprises increasing IFN-γ-producing CD8⁺ T cell populations in the subject by administering to the subject the compound of any one of claims 1-9 or the pharmaceutical composition of claim 10.

39. The method of any one of claims 26-38, wherein the preventing or slowing the development of the cancer comprises decreasing myeloid-derived suppressor cell (MDSC) populations in the subject by administering to the subject the compound of any one of claims 1-9 or the pharmaceutical composition of claim 10.

40. The method of any one of claims 12-25 and 27-39, wherein the α-PD-L1 checkpoint inhibitor is selected from the group consisting of atezolizumab, avelumab, durvalumab, KN035, cosibelimab, AUNP12, CA-170, and BMS-986189.

41. The method of any one of claims 12-25 and 27-39, wherein the α-PD-L1 checkpoint inhibitor is selected from the group consisting of atezolizumab, avelumab, and durvalumab.
